(19) United States
(12) Patent Application Publication
(10) Pub. No.: US 2005/0221354 A1
(43) Pub. Date: Oct. 6, 2005

(54) Nucleic acid arrays for monitoring expression profiles of drug target genes

(75) Inventor: William Martin Mounts, Andover, MA (US)

Correspondence Address: NIXON PEABODY LP, 401 9TH STREET, N.W., SUITE 900, WASHINGTON, DC, 20004, US

(73) Assignee: Wyeth, Madison, NJ (US)

(21) Appl. No.: 11/060,756

(22) Filed: Feb. 18, 2005

Related U.S. Application Data
(60) Provisional application No. 60/545,213, filed on Feb. 18, 2004.

(51) Int. Cl. C12Q 1/68; C12M 1/34

(52) U.S. Cl. 435/6; 435/287.2

(57) ABSTRACT

The present invention provides nucleic acid arrays and methods of using the same for detecting or monitoring expression profiles of drug target genes. Non-limiting examples of drug target genes include kinase genes, phosphatase genes, protease genes, G-protein coupled receptor genes, nuclear hormone receptor genes, and ion channel genes. The present invention also provides methods of using nucleic acid arrays for the identification or validation of drugs or drug targets. In one embodiment, a nucleic acid array of the present invention is concentrated with probes for drug target genes. These probes constitute a substantial portion of all of the polynucleotide probes that are stably attached to the nucleic acid array, and can hybridize under stringent or nucleic acid array hybridization conditions to the tiling sequences selected from Attachment C, or the complements thereof.

### RELATED APPLICATIONS

This application claims the benefit and incorporates by reference the entire disclosure of U.S. Provisional Application Ser. No. 60/545,213, entitled “Nucleic Acid Arrays for Monitoring Expression Profiles of Drug Target Genes,” which was filed on Feb. 18, 2004.

All materials on the compact discs labeled “Copy 1” and “Copy 2” are incorporated herein by reference in their entireties. Each of the compact discs includes the following files: “Attachment A—Consensus Sequences.txt” (208 KB, created Jan. 7, 2004), “Attachment B—Exemplar Sequences.txt” (560 KB, created Jan. 7, 2004), “Attachment C—Tiling Sequences.txt” (759 KB, created Jan. 7, 2004), “Attachment D—Location of Tiling Sequences in Corresponding Parent Sequences.txt” (163 KB, created Jan. 8, 2004), “Attachment E—Gene Class.txt” (100 KB, created Jan. 8, 2004), “Attachment F—Probes.txt” (5,473 KB, created Jan. 8, 2004), “Attachment G—Probes.txt” (9,132 KB, created Jan. 7, 2004), and “Sequence Listing.ST25.txt” (51,515 KB, created Jan. 21, 2005).

### TECHNICAL FIELD

The present invention relates to nucleic acid arrays and methods of using the same for detecting or monitoring expression profiles of drug target genes. The present invention also relates to methods for the identification or validation of drugs or drug targets.

### BACKGROUND

Numerous assays are available for evaluating the effects of drug candidates on gene expression. These assays, however, frequently generate excessive information that is irrelevant to the actions of drug candidates. For example, an Affymetrix Human Genome U133 Set contains probe sets for approximately 33,000 human genes. A global gene expression analysis using this microarray may generate hundreds if not thousands of genes whose expression profiles appear to be modulated by a drug candidate. Many of these genes, however, have little therapeutic value, and the identification and removal of these genes are laborious and time-consuming. Moreover, whole genome microarrays are expensive, and the number of probes for each transcript on a whole genome microarray is often limited, compromising the reliability and reproducibility of probe set signal values.

### SUMMARY OF THE INVENTION

The present invention features nucleic acid arrays that are concentrated with probes for drug target genes. The manufacturing costs of these nucleic acid arrays can be significantly less than those of traditional whole genome microarrays. The sizes of these nucleic acid arrays can also be reduced, resulting in less sample usage and lower reagent costs per experiment. In addition, the number of probes for each transcript on a nucleic acid array of the present invention can be significantly increased, leading to a more robust overall probe set signal value and substantially improving the reliability and reproducibility of the detection. All of these features allows for cost-effective expression profiling of drug target genes, thereby facilitating the process of drug discovery and development.

In one aspect, a substantial portion of all polynucleotide probes on a nucleic acid array of the present invention consists of probes for drug target genes. These probes can hybridize under stringent or nucleic acid array hybridization conditions to the RNA transcripts, or the complements thereof, of drug target genes. Examples of drug target genes that are amenable to the present invention include, but are not limited to, kinase genes, phosphatase genes, protease genes, G-protein coupled receptor genes, nuclear hormone receptor genes, and ion channel genes.

In one embodiment, the drug target gene probes constitute at least 15%, 20%, 25%, 30%, 35%, 40%, 45%, 50%, or more of all of the polynucleotide probes that are stably attached to a nucleic acid array of the present invention. In another embodiment, the drug target gene probes constitute at least 30%, 40%, 50%, 60%, 70%, 80%, 90%, or 100% of all of the perfect match probes that are stably attached to a nucleic acid array of the present invention. In many instances, a nucleic acid array of the present invention also includes mismatch probes for each drug target gene probe on the array.

In still another embodiment, multiple classes of drug target genes can be detected by a nucleic acid array of the present invention, and the nucleic acid array includes at least 1, 2, 5, 10, 20, 30, 40, 50, 100, 200, 300, 400, 500, or more probes for each class of drug target genes. In one example, the nucleic acid array includes one or more probes for an ADAMTS4 gene which encodes a disintegrin-like and metalloprotease with thrombospondin type 1 motif, 4.

In still yet another embodiment, a nucleic acid array of the present invention includes at least 1, 2, 5, 10, 50, 100, 150, 200, 250, 500, 1,000, 2,000, 3,000 or more probes, each of which is capable of hybridizing under stringent or nucleic acid array hybridization conditions to a different respective tiling sequence selected from Attachment C (SEQ ID NOs: 4,273-8,544), or the complement thereof. In one example, a nucleic acid array of the present invention includes at least one probe for each tiling sequence selected from Attachment C, or the complement thereof.

In yet another embodiment, a nucleic acid array of the present invention includes at least 1, 2, 5, 10, 15, 20, 25, 30, 35, 40, 45, 50, 55, 60, 65, 70, or more probes for each drug target gene or tiling sequence of interest. In one example, a nucleic acid array of the present invention includes at least from 35 to 68 probes for each drug target gene or tiling sequence of interest.

In still another embodiment, a nucleic acid array of the present invention includes at least 1, 2, 5, 10, 50, 100, 150, 200, 250, 500, 1,000, 2,000, 3,000 or more probes selected from Attachment F (SEQ ID NOs: 8,647-116,337), or the complements thereof In one example, the nucleic acid array includes each and every probe selected from Attachment F, or the complement thereof.

The nucleic acid arrays of the present invention can include 1, 2, 3, 4, 5, or more substrate supports. In one embodiment, all of the polynucleotide probes on a nucleic acid array are attached to a single substrate support. In another embodiment, the nucleic acid array is a bead array which includes numerous beads that are stably associated with probes for drug target genes.

In another aspect, the present invention provides methods for the detection, identification, or evaluation of agents that are capable of modulating expression profiles of drug target genes. These methods include the steps of contacting one or more cells with a candidate molecule, preparing a nucleic acid sample from the cell(s), and hybridizing the nucleic acid sample to a nucleic acid array of the present invention to detect any change in hybridization signals before and after the contact. A change in the hybridization signals is indicative that the candidate compound is capable of modulating the expression profiles of drug target genes.

In one embodiment, the change in the hybridization signals is a result of modulation of the transcription or translation of a drug target gene of interest. The change in the hybridization signals can also be a result of modulation of the expression or function of another gene or gene product, which in turn alters the expression of the drug target gene.

The agents identified by the present invention can be used to teat mammals in need thereof. In many instances, a drug target gene is abnormally expressed in a mammal that is to be treated, and an agent capable of modulating the expression profile of the drug target gene is identified and used to correct or alleviate the abnormality in the mammal.

In yet another aspect, the present invention provides other methods for the detection, identification, or evaluation of agents that are capable of modulating expression profiles of drug target genes. These methods include the steps of administering a candidate molecule to a human or animal, preparing a nucleic acid sample from the human or animal, and hybridizing the nucleic acid sample to a nucleic acid array of the present invention to detect any change in hybridization signals before and after the administration. A change in the hybridization signals is indicative that the candidate compound is capable of modulating the expression profiles of drug target genes in the human or animal.

In still yet another aspect, the present invention provides methods for making nucleic acid arrays. The methods include the steps of (1) selecting numerous polynucleotides, each polynucleotide capable of hybridizing under stringent or nucleic acid array hybridization conditions to a different respective drug target gene, and (2) stably attaching the selected polynucleotides to one or more substrate supports to create a nucleic acid array, where the selected polynucleotides constitute a substantial portion of all of the polynucleotide probes that are stably attached to the nucleic acid array.

The present invention also features protein arrays for detecting or-monitoring expression profiles of drug target genes. Each protein array of the present invention includes probes which can specifically bind to protein products of human drug target genes. In one embodiment, the probes on a protein array of the present invention are antibodies. In another embodiment, a substantial portion of all of the probes on a protein array of the present invention consists of antibodies for the protein products encoded by the parent sequences selected from Attachments A or B.

In addition, the present invention also features polynucleotide collections. In one embodiment, a polynucleotide collection includes at least 1, 2, 3, 4, 5, 10, 20, 50, 100, 200, 500, 1,000, or more probes capable of hybridizing under stringent or nucleic acid array hybridization conditions to the corresponding tiling sequences selected from Attachment C, or the complements thereof. In another embodiment, the polynucleotide collection includes at least 1, 2, 3, 4, 5, 10, 20, 50, 100, 200, 500, 1,000, or more tiling sequences selected from Attachment C, or the complements thereof. In yet another embodiment, a polynucleotide collection includes at least 1, 2, 3, 4, 5, 10, 20, 50, 100, 200, 500, 1,000, or more sequences selected from SEQ ID NOs: 1-4,272, or the complements thereof.

Other features, objects, and advantages of the present invention are apparent in the detailed description that follows. It should be understood, however, that the detailed description, while indicating preferred. embodiments of the invention, is given by way of illustration only, not limitation. Various changes and modifications within the scope of the invention will become apparent to those skilled in the art from the detailed description.

### DETAILED DESCRIPTION

The present invention provides nucleic acid arrays that are concentrated with probes for drug target genes. In one embodiment, a substantial portion of all of the polynucleotide probes that are stably attached to a nucleic acid array of the present invention consists of probes for drug target genes. These probes can hybridize under stringent or nucleic acid array hybridization conditions to the RNA transcripts, or the complements thereof, of drug target genes. Exemplary drug target genes include, but are not limited to, genes that encode kinases, phosphatases, proteases, G-protein coupled receptors, nuclear hormone receptors, or ion channels. In another embodiment, a substantial portion of all of the polynucleotide probes that are stably attached to a nucleic acid array of the present invention is capable of hybridizing under stringent or nucleic acid array hybridization conditions to the tiling sequences selected from Attachment C (SEQ ID NOs: 4,273-8,544), or the complements thereof. The nucleic acid arrays of the present invention can also include probes for non-drug target genes.

In many cases, the use of the nucleic acid arrays of the present invention reduces or eliminates the painstaking process for identifying or removing signals that are not associated with drug target genes. In addition, by using a less number of genes, the present invention reduces the costs associated with the manufacture and use of nucleic acid arrays.

The following sections focus on the creation of nucleic acid arrays that are suitable for detecting or monitoring the expression profiles of human drug target genes. As appreciated by those skilled in the art, the same methodology can be readily adapted to making nucleic acid arrays for expression profiling of animal drug target genes. Exemplary animals include, but are not limited to, primates, rodents, rabbits, canines, nematode worms, fruit flies, and frogs. A nucleic acid array of the present invention can also include probes for drug target genes of different species (e.g., including probes for human drug target genes as well as probes for animal drug target genes). As used herein, the term “or” means “and/or” unless stated otherwise.

A. Collection of mRNA, cDNA, and other Coding or Non-Coding Sequences of Human Drug Target Genes

Drug target genes include genes whose functions or expressions can be modified by drugs. As used herein, a drug can be any compound of any degree of complexity that is capable of modulating a biological system to achieve a desirable effect, whether by known or unknown mechanisms and whether used therapeutically or not. Examples of drugs include, but are not limited to, small molecules with therapeutic effects; naturally-occurring factors or their analogs, such as endocrine, paracrine, or autocrine factors, or factors interacting with cell receptors of all types; and intracellular factors or their analogs, such as elements of intracellular signaling pathways. The biological effect of a drug can be, without limitation, a consequence of drug-mediated changes in the rate or extent of transcription or translation of one or more genes, the degradation or processing of one or more RNA transcripts, the degradation or post-translational modification of one or more proteins, or the inhibition or stimulation of the action or activity of one or more proteins.

Examples of drug target genes that can be assessed according to the present invention include, but are not limited to, kinase genes, phosphatase genes, protease genes, G-protein coupled receptor genes, nuclear hormone receptor genes, and ion channel genes. These genes have been the major targets for drug action and development. Many products of these genes play important roles in intercellular communication and signal transduction pathways.

Protein kinases are key components of many signal transduction pathways. Malfunctions of protein kinases have been associated with a wide variety of diseases. A large number of therapeutic strategies have been based on compounds that can activate or inactivate specified protein kinases. For instance, a year 2002 survey of ongoing clinical trials in the United States showed that more than 100 clinical trials involved the modulation of kinases. These clinical trials were directed to a broad spectrum of therapeutic indications, including asthma, Parkinson's, inflammation, psoriasis, rheumatoid arthritis, spinal cord injuries, muscle conditions, osteoporosis, graft versus host disease, cardiovascular disorders, autoimmune disorders, retinal detachment, stroke, epilepsy, ischemia/reperfusion, breast cancer, ovarian cancer, glioblastoma, non-Hodgkin's lymphoma, colorectal cancer, non-small cell lung cancer, brain cancer, Kaposi's sarcoma, pancreatic cancer, liver cancer, and other tumors. Numerous modulators of kinase activities have been investigated in clinical trials. They include, for example, antisense oligonucleotides, antibodies, naturally-occurring molecules or their analogs, and other DNA/RNA molecules that are used in gene-based or RNA-based therapies.

Protein kinases include, but are not limited to, cAMP dependent protein kinases (PKAs), Ca²⁺ and phospholipid-dependent protein kinases (PKCs), mitogen-activated protein (MAP) kinases, Ca²⁺/calmodulin dependent protein kinases, cyclin-dependent kinases (CDKs), DNA-dependent protein kinase (DNA-PK), and protein tyrosine kinases (PTKs). cAMP dependent protein kinases (PKAs) are a family of serine/threonine kinases. Altered PKA expression has been implicated in numerous disorders or diseases, including tumors, thyroid disorders, diabetes, atherosclerosis, and cardiovascular diseases. Known regulators of PKAs include various inhibitors or activators of adenylate cyclase, which controls cAMP levels in cells.

Ca²⁺ and phospholipid-dependent protein kinases (PKCs) are another family of serine/threonine kinases. PKCs have been implicated in cell proliferation and differentiation, apoptosis, neurotransmission, and long-term potentiation. Changes in PKC activities have also been implicated in hyperglycemia and the vascular complications of diabetes. Different isoforms of PKC require different activation requirements. For instance, activation of isoforms α, β and γ requires phosphatidylserine, diacylglycerol, and Ca²⁺, while activation of other isoforms may require phosphatidylserine or diacylglycerol, but not Ca²⁺.

Mitogen-activated protein (MAP) kinases are also a family of serine/threonine kinases. MAP kinases mediate signal transduction to the nucleus in response to diverse extracellular stimuli. They also regulate intracellular signaling pathways. MAP kinases have been implicated in inflammation, apoptosis, cell proliferation and differentiation, response to cellular stress, and carcinogenesis. At least three different MAP kinase signal transduction pathways have been identified. They are the ERK1/2 mediated pathway, the JNK/SAPK mediated pathway, and the p38 kinase mediated pathway. The extracellular stimuli capable of activating the MAP kinase signaling pathways include epidermal growth factor (EGF), ultraviolet radiation, hyperosmolar medium, heat shock, endotoxic lipopolysaccharide (LPS), insulin, insulin-like growth factor 1 (IGF-1), and pro-inflammatory cytokines, such as tumor necrosis factor (TNF) and interleukin-1 (IL-1).

Ca²⁺/calmodulin dependent protein kinases are involved in regulation of smooth muscle contraction (e.g., MLC kinase), glycogen breakdown (e.g., phosphorylase kinase), and neurotransmission (e.g., CaM kinase I and. CaM kinase II). These kinases are members of a serine/threonine kinase family. They can phosphorylate a variety of substrates, such as synapsin I and II, the cystic fibrosis conductance regulator protein, CFTR (a chloride ion channel that is defective in patients with cystic fibrosis), CREB (cAMP responsive element binding protein), and other transcription factors. The deletion of Ca²⁺/calmodulin dependent protein kinase II in mice produces behavioral abnormalities including increased defensive aggression and decreased fear response. In addition, it has been indicated that Ca²⁺/calmodulin dependent protein kinases are required for long-term potentiation. For example, mice with mutated Ca²⁺/calmodulin dependent protein kinase II have impaired learning ability.

Cyclin-dependent kinases (CDKs) are a family of serine/threonine kinases that control the mitotic process. Exemplary members of this family include cdc2 which regulates the transition from G2 to M phase. Activation of CDKs requires multiple inputs. In addition to the binding of cyclin, CDK activation requires phosphorylation as well as dephosphorylation of specific residues. CDK defects have been observed in various cancer phenotypes, making CDKs important targets for new cancer treatment drugs.

DNA-dependent protein kinases (DNA-PKs) are nuclear serine/threonine kinases. They have been implicated in double-stranded DNA repair and protection, modification of chromatin structure, and maintenance of telomeres. Cells with defective DNA-PK activities lack the ability to repair radiation-induced DNA breaks and therefore are sensitive to ultraviolet and ionizing radiation. Moreover, it has been shown that inhibition of DNA-PK can increase the efficacy of anti-tumor treatment with radiation or chemotherapeutic agents.

Protein tyrosine kinases (PTKs) are involved in numerous cellular events, including cell proliferation and differentiation, apoptosis, cell cycle regulation, signal transduction from extracellular stimuli to intracellular targets, T cell activation, B cell activation, and hematopoiesis. PTK defects or deficiencies have been implicated in a wide range of diseases, such as breast cancer, prostate cancer, leukemia, glioma, squamous cell carcinoma, colon cancer, multiple endocrine neoplasia, medullary thyroid cancer, epithelial cell cancer, Hirschsprung's disease, Bruton's disease, psoriasis, diabetes, and autoimmune diseases. PTKs can be divided into two classes—namely, the transmembrane PTKs and the non-transmembrane PTKs.

The transmembrane PTKs are receptors for most growth factors. Binding of a growth factor to a receptor PTK can activate the PTK or other proteins by phosphorylation. Growth factors capable of activating receptor PTKs include, but are not limited to, epidermal growth factor (EGF), platelet-derived growth factor (PDGF), fibroblast growth factor (FGF), hepatocyte growth factor (HGF), insulin and insulin-like growth factors (IGF), nerve growth factor (NGF), vascular endothelial growth factor (VEGF), and macrophage colony stimulating factor (CSF).

In addition to the above-described kinases, other protein kinase genes can also be the potential drug targets. These protein kinases include, for example, cGMP-dependent kinases, 5′-AMP-activated protein kinases (AMPK), and proliferation-related kinase (PRK). Like PKAs, cGMP-dependent kinase is a second-message-dependent serine/threonine kinase. Its activity is modulated by the cellular cGMP levels. cGMP-dependent kinase has been implicated in the regulation of smooth muscle relaxation, platelet function, sperm metabolism, and nucleic acid synthesis. AMPK is a regulator of fatty acid and sterol synthesis. AMPK mediates the response to cellular stress, such as heat shock or depletion of glucose or ATP. PRK is a serum/cytokine inducible kinase. It is involved in the regulation of cell cycle and cell proliferation. PRK has been considered a potential proto-oncogene whose deregulation in normal tissues may lead to oncogenic transformation.

As a counterpart of protein kinases, protein phosphatases reverse the protein phosphorylation process by protein kinases. The levels of protein phosphorylation required for normal cell growth and differentiation are achieved through the coordinated action of protein kinases and phosphatases. Depending on the cellular context, these two types of enzymes may either antagonize or cooperate with each other during signal transduction. An imbalance between these enzymes may impair normal cell functions, leading to metabolic disorders or cellular transformation.

Protein phosphatases can be roughly divided into three families—namely, serine/threonine phosphatases, tyrosine phosphatases, and dual-specificity phosphatases. Serine/threonine phosphatases are either cytosolic or associated with a receptor. On the basis of their sensitivity to two thermostable proteins (i.e., inhibitors 1 and 2) and their divalent cation requirements, the serine/threonine phosphatases can be divided into at least four distinct groups. They are PP1, PP2A, PP2B, and PP2C.

PP1 dephosphorylates many of the proteins phosphorylated by cAMP-dependent protein phosphatase and therefore is an important regulator of cAMP-mediated signal transduction pathways. PP2A is a main phosphatase responsible for reversing the phosphorylation of serine/threonine phosphatases. PP2A have been implicated in metabolism, transcription and translation regulation, RNA splicing, cell differentiation, cell cycle, and oncogenic transformation. PP2B, also known as calcineurin, is a Ca²⁺-activated phosphatase. PP2B is involved in a variety of cellular functions, including ion channel regulation, neurotransmission, transcription regulation, muscle glycogen metabolism, and lymphocyte activation. PP2C is an Mg²⁺-dependent phosphatase that plays a role in the regulation of cAMP-activated protein phosphatase activity, Ca²⁺-dependent signal transduction, tRNA splicing, and signal transduction related to heat shock responses.

Protein tyrosine phosphatases (PTPs) are involved in cell differentiation and malignant transformation. PTP targets include receptors, transcription factors, ion channels, cellular motors, and certain structural proteins such as filaments.

Dual-specificity phosphatases (DSPs) regulate mitogenic signal transduction pathways. DSPs may also be involved in meiosis and spermatogenesis.

The importance of phosphatases in the etiology of diseases has been well established. Malfunction of phosphatases has been associated with numerous human diseases or disorders, including renal and small lung carcinoma, Charcot-Marie-Tooth disease type 4B1, allergy, asthma, obesity, myocardial hypertrophy, and Alzheimer's disease.

Another class of drug target genes is protease. Proteases are involved in a wide variety of biological processes, including post-translational modifications, blood coagulation, fibrinolysis, complement activation, fertilization, hormone production, degradation of undesirable proteins or invading organisms, tumor metastasis, stress response, wound healing, tissue remodeling, cell proliferation and differentiation, and other signal transduction pathways. Proteases include endopeptidases and exopeptidases. Endopeptidases cleave peptide bonds at points within a protein, and exopeptidases remove amino acids sequentially from either N or C-terminus of a protein. At least four mechanistic classes of endopeptidases have been recognized. They are the aspartic, serine, metallo, and cysteine proteinases.

The aspartic proteinases include at least one active aspartate residue at the catalytic center. Catalysis by aspartic proteases involves the formation of a non-covalent neutral tetrahedral intermediate. Examples of the aspartic proteases include pepsin A, presenilin 1, chymosin, Iysosomal cathepsins D, renin, and retropepsin (from human immunodeficiency virus type 1).

The serine proteinases include trypases (cleaving arginine or lysine), aspases (cleaving after aspartate), chymases (cleaving after phenylalanine or leucine), metases (cleaving after methionine), and serases (cleaving after serine). The serine proteases are so named because of the presence of a serine residue in their catalytic sites.

The metallo proteinases differ widely in sequences and structures. Many of the metallo proteinases contain a zinc atom in their catalytic sites. Examples of the metallo proteinases include membrane alanyl aminopeptidase, germinal peptidyl-dipeptidase A, collagenase 1, neprilysin, carboxypeptidase A, membrane dipeptidase, and S2P protease.

The cysteine proteinases contain a cysteine nucleophile at the catalytic site. Like the serine proteinases, catalysis by cysteine proteinases involves the formation of a covalent intermediate between the substrate and the active-site cysteine. Exemplary cysteine proteinases include cytosolic calpains and lysosomal cathepsins.

Uncontrolled protease activity has been implicated in many diseases, such as arteriosclerosis, muscular dystrophy, amyotrophy, rheumatoid arthritis, osteoarthritis, autoimmune diseases, inflammation, infection, cancer, and degenerative disorders. Therefore, proteases have been the major targets for drug action and development.

G-protein coupled receptors (GPCR) are a superfamily of integral membrane proteins which transduce extracellular signals. GPCRs include receptors for biogenic amines, such as dopamine, epinephrine, histamine, glutamate (metabotropic effect), acetylcholine (muscarinic effect), and serotonin; for lipid mediators of inflammation such as prostaglandins, platelet activating factor, and leukotrienes; for peptide hormones such as calcitonin, C5a anaphylatoxin, follicle stimulating hormone, gonadotropin releasing hormone, neurokinin, oxytocin, and thrombin; and for sensory signal mediators, such as retinal photopigments and olfactory stimulatory molecules.

A typical GPCR has seven transmembrane domains. A GPCR becomes activated when it binds to an extracellular ligand. The interaction between the ligand and the GPCR changes the binding affinity of the GPCR to the coupled G-protein, which in turn enables GTP to bind with enhanced affinity to the G-protein. This allows the G-protein to activate the downstream second messenger generator(s), such as adenylate cyclase. The activation of the second messenger generator(s) alters the cellular level of the respective second messenger molecule(s), thereby triggering the next effector in the signaling cascade. Exemplary second messengers include cAMP, cGMP, inositol trisphosphate, diacylglycerol, and Ca²⁺. Activity of GPCRs can be regulated by phosphorylation of the intracellular or extracellular domains of the receptor.

GPCR-mediated signal transduction pathways have been implicated in a variety of diseases, including, for example, hypotension, hypertension, angina pectoris, myocardial infarction, depression, delirium, dementia, severe mental retardation, asthma, Parkinson's disease, acute heart failure, urinary retention, osteoporosis, and cancers. Thus, GPCRs represent another platform for drug discovery.

Nuclear hormone receptor is a large family of ligand-activated transcription factors that modifies the expression of target genes by binding to specific cis-acting sequences. Nuclear hormone receptors include both orphan receptors and receptors for a wide variety of clinically significant ligands including glucocorticoids, androgens, mineralocorticoids, progestins, estrogens, thyroid hormones, vitamin D, retinoids, peroxisomes, and icosanoids.

A typical nuclear hormone receptor has a variable N-terminal region, a conserved DNA-binding domain, a variable hinge region, a conserved ligand binding domain, and a variable C-terminal region. Ligand binding can induce a conformational change in the receptor and promote its association with transcriptional coactivators. The resulting complex can bind to the target DNA sequence with increased affinity.

Nuclear hormone receptors have been implicated in numerous disorders or diseases. They include, but are not limited to, Parkinson's disease, adrenal hypoplasia, hypogonadism, hypercholesterolemia, obesity, diabetes, infertility, central nerve system disorders, sleep disorders, immune disorders, metabolic disorders, and tumors. Thus, nuclear hormone receptors represent another class of primary targets for drug discovery.

Ion channels are transmembrane proteins that regulate the flow of ions across cellular membranes. Ion channels participate in diverse biological processes, including the generation and timing of action potentials, synaptic transmissions, secretion of hormones, contraction of muscles, and intercellular communication. Ion channels exist in vivo in multimeric forms and comprise pore-forming and auxiliary subunits. These subunits are coded by several distinct gene families. Ion channel properties can be modulated by second messenger cascades. Some ion channels can directly interact with intracellular or membrane proteins, such as protein kinases, G-proteins, and cytoskeleton-associated proteins.

Exemplary ion channels include extracellular ligand-gated channels (e.g., neurotransmitter gated channels), intracellular ligand-gated channels (e.g., cyclic nucleotide or calcium gated channels), voltage-gated channels (e.g., potassium, sodium, or calcium channels), inward rectifier (e.g., inward rectifier K⁺ Channel), gap junction channels (e.g., channels formed by connexins or desmosome), ATP gated channels, and proton-gated channels.

Many diseases are known to be associated with the dysfunction of ion channels. These diseases include, for example, cardiac arrhythmias, angina pectoris, cystic fibrosis, myotonia, epilepsy Alzheimer's disease, Parkinson's disease, long QT syndrome, sick sinus syndrome, age-related memory loss, and sudden death syndrome. A better understanding of the ion channel expression and its regulation would lead to the discovery of new drugs capable of treating ion channel related diseases.

mRNA, cDNA, and other protein coding or non-coding sequences of human drug target genes can be collected from a variety of sources, such as GenBank and GENESEQ™ (Derwent). These sequence databases include a large number of EST and cDNA sequences, and many of these sequences are annotated. Sequences encoding drug target genes can therefore be identified according to their annotations.

These sequence databases also contain an enormous amount of human genomic sequences. Open reading frames (ORFs) in these genomic sequences can be predicted or isolated using methods known in the art. Suitable methods for this purpose include, but are not limited to, GeneMark (provided by the European Bioinformatics Institute), Glimmer (provided by The Institute for Genome Research), and ORF Finder (provided by the National Center for Biotechnology Information (NCBI)). The ORFs that encode or share high sequence homology with known human target genes can be identified. The functions of the polypeptides encoded by these identified ORFs can be analyzed using standard methods, such as cell-based assays or in vitro transcription/translation systems.

Human drug target gene sequences collected using the above-described methods, together with sequences collected from other sources, can be clustered to identify highly homologous sequences. Suitable clustering algorithms include, but are not limited to, the CAT (cluster and alignment tool) software package provided by DoubleTwist. See Clustering and Alignment Tools User's Guide (DoubleTwist, Inc., 2000).

The CAT program can reduce the redundancy, as well as mask low-complexity regions, of the input sequence set. The resulting sequence set derived from CAT contains two distinct groups of sequences. The first group is a set of consensus sequences derived from multiple sequence alignment which is produced from CAT sub-clusters containing more than one sequence. These multi-sequence sub-clusters may also include single transcripts represented in the input sequence set numerous times. The second group is a set of exemplar sequences that do not cluster with any other CAT sub-cluster. The consensus and exemplar sequences can be generated such that any base ambiguity is identified with the respective IUPAC (International Union of Pure and Applied Chemistry) or WIPO Standard ST.25 (1998) base representation.

In a small number of cases, the multi-sequence sub-clusters may contain a large number of sequences due to clustering artifacts (e.g., highly homologous genes or domains). In these cases, through more stringent clustering parameters, the large sub-clusters can be re-clustered. In addition, the consensus sequences can be manually curated to verify cluster membership.

Examples of the consensus sequences obtained using the above-described method are depicted in SEQ ID NOs: 1-1,087. Examples of the exemplar sequences so obtained are shown in SEQ ID NOs: 1,088-4,272. Each consensus or exemplar sequence has a header which includes a qualifier (after “wyeHumanDT1a”) and other information of the sequence. These headers are illustrated in Attachment A for the consensus sequences and Attachment B for the exemplar sequences, respectively. As used herein, the consensus and exemplar sequences are collectively referred to as the “parent sequences.”

Attachment E shows the gene class of each parent sequence. These gene classes include the kinase gene class (“Kinase”), the phosphatase gene class (“Phosphatase”), the protease gene class (“Protease”), the G-protein coupled receptor gene class (“GPCR”), the nuclear hormone receptor gene class (“NHR”), and the ion channel gene class (“Ion Channel”).

mRNA, cDNA, or other protein coding or non-coding sequences of drug target genes can also be obtained by sequencing cDNA libraries. This method is particularly useful for the identification of drug target genes that are specifically expressed in certain tissue or tissues. cDNA libraries suitable for this purpose can be derived from any human tissue, such as heart, liver, kidney, brain, lung, pancreas, spleen, blood, muscle, bone, cartilage, or bone marrow. Methods for constructing cDNA libraries from a tissue of interest are well known in the art. Non-limiting examples of commercial kits suitable for this purpose include the CloneMiner™ cDNA Library Construction Kit provided by Invitrogen (Carlsbad, Calif.).

cDNA clones in a library can be readily sequenced using any method known in the art. In a standard method, the cDNA insert in a clone can be sequenced using primers designed from the common vector sequences adjacent to the 5′ or 3′ end of the cDNA inserts. These 5′ or 3′ reads from a cDNA library can be compared to the protein coding sequences of known kinases, phosphatases, proteases, G-protein coupled receptors, nuclear hormone receptors, and ion channels. cDNA clones sharing high sequence homology with known drug target genes can therefore be identified. These clones can be further analyzed to determine if they encode functional drug target genes.

In another embodiment, function-based library screen is employed to identify sequences having drug target gene activities. Libraries suitable for this purpose include cDNA libraries or peptide libraries (e.g., phage-displayed peptide libraries or synthetic peptide libraries).

The human drug target genes thus identified, together with sequences collected from available sequence databases or other sources, can be clustered using CAT or other programs to derive consensus or exemplar sequences. As appreciated by those skilled in the art, mRNA, cDNA, or other protein coding or non-coding sequences of drug target genes can be similarly collected from animals. Consensus and exemplar sequences can be generated from these sequences using the methods described above.

B. Preparation of Polynucleotide Probes for Human Drug Target Genes

The parent sequences depicted in Attachments A and B (SEQ ID NOs: 1-4,272) can be used to prepare polynucleotide probes for human drug target genes. These probes can hybridize under stringent or nucleic acid array hybridization conditions to the RNA transcripts, or the complements thereof, of human drug target genes (e.g., mRNA, cRNA, or cDNA). In many embodiments, a probe for a drug target gene is incapable of hybridizing under stringent or nucleic acid array hybridization conditions to the RNA transcripts, or the complements thereof, of other genes (including other drug target genes).

As used herein, “nucleic acid array hybridization conditions” refer to the temperature and ionic conditions that are normally utilized for nucleic acid array hybridization. For instance, these conditions can include 16-hour hybridization at 45° C., followed by at least three 10-minute washes at room temperature. The hybridization buffer can include 100 mM MES, 1 M [Na⁺], 20 mM EDTA, and 0.01% Tween 20. The pH of the hybridization buffer preferably is between 6.5 and 6.7. The wash buffer is 6×SSPET. 6×SSPET contains 0.9 M NaCl, 60 mM NaH₂PO₄, 6 mM EDTA, and 0.005% Triton X-100. Under more stringent nucleic acid array hybridization conditions, the wash buffer can include 100 mM MES, 0.1 M [Na⁺], and 0.01% Tween 20.

“Stringent conditions” are at least as stringent as, for example, conditions G-L in Table 1. In certain embodiments, highly stringent conditions A-F in Table 1 can be used. In Table 1, hybridization is carried out under the hybridization conditions (Hybridization Temperature and Buffer) for about four hours, followed by two 20-minute washes under the corresponding wash conditions (Wash Temp. and Buffer).

TABLE 1
Stringency Conditions

| | Poly- | Hybrid | | |
|---|---|---|---|---|
| Stringency | nucleotide | Length | Hybridization | Wash Temp. |
| Condition | Hybrid | (bp)¹ | Temperature and Buffer^{H} | and Buffer^{H} |
| A | DNA:DNA | >50 | 65° C.; 1 × SSC -or- | 65° C.; 0.3 × SSC |
| | | | 42° C.; 1 × SSC, | |
| | | | 50% formamide | |
| B | DNA:DNA | <50 | T_{B}*; 1 × SSC | T_{B}*; 1 × SSC |
| C | DNA:RNA | >50 | 67° C.; 1 × SSC -or- | 67° C.; 0.3 × SSC |
| | | | 45° C.; 1 × SSC, | |
| | | | 50% formamide | |
| D | DNA:RNA | <50 | T_{D}*; 1 × SSC | T_{D}*; 1 × SSC |
| E | RNA:RNA | >50 | 70° C.; 1 × SSC -or- | 70° C.; 0.3 × SSC |
| | | | 50° C.; 1 × SSC, | |
| | | | 50% formamide | |
| F | RNA:RNA | <50 | T_{F}*; 1 × SSC | T_{f}*; 1 × SSC |
| G | DNA:DNA | >50 | 65° C.; 4 × SSC -or- | 65° C.; 1 × SSC |
| | | | 42° C.; 4 × SSC, | |
| | | | 50% formamide | |
| H | DNA:DNA | <50 | T_{H}*; 4 × SSC | T_{H}*; 4 × SSC |
| I | DNA:RNA | >50 | 67° C.; 4 × SSC -or- | 67° C.; 1 × SSC |
| | | | 45° C.; 4 × SSC, | |
| | | | 50% formamide | |
| J | DNA:RNA | <50 | T_{J}*; 4 × SSC | T_{J}*; 4 × SSC |
| K | RNA:RNA | >50 | 70° C.; 4 × SSC -or- | 67° C.; 1 × SSC |
| | | | 50° C.; 4 × SSC, | |
| | | | 50% formamide | |
| L | RNA:RNA | <50 | T_{L}*; 2 × SSC | T_{L}*; 2 × SSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}SSPE (1 × SSPE is 0.15 M NaCl, 10 mM NaH₂PO₄, and 1.25 mM EDTA, pH 7.4) can be substituted for SSC (1 × SSC is 0.15 M NaCl and 15 mM sodium citrate) in the hybridization and wash buffers. | | | | |
| T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). | | | | |
| # For hybrids between 18 and 49 base pairs in length, Tₘ(° C.) = 81.5 + 16.6(log₁₀Na⁺) + 0.41(% G + C) − (600/N), where N is the number of bases in the hybrid, and Na⁺ is the molar concentration of sodium ions in the hybridization buffer (Na⁺ for 1 × SSC = 0.165 M). | | | | |

In many embodiments, the polynucleotide probes for each drug target gene can hybridize under stringent or nucleic acid array hybridization conditions to the corresponding parent sequence of the gene, or the full complement thereof. Where a parent sequence contains one or more ambiguous residues (i.e., residue “n”), the probes for that parent sequence can be designed such that they are capable of hybridizing under stringent or nucleic acid array hybridization conditions to an unambiguous segment of the parent sequence, or the complement of the unambiguous segment. In one example, each probe for a parent sequence comprises or consists of an unambiguous sequence fragment of that parent sequence, or the complement thereof. In one embodiment, the probes for a drug target gene or a parent sequence are incapable of hybridizing under stringent or nucleic acid array hybridization conditions to other drug target genes or parent sequences.

The length of each polynucleotide probe employed in the present invention can be selected to produce the desired hybridization effects. For example, the probes can include or consist of at least 10, 15, 20, 25, 30, 35, 40, 45, 50, 60, 70, 80, 90, 100, 200, 300, 400 or more consecutive nucleotides. The probes can be DNA, RNA, or PNA. Other modified forms of DNA, RNA, or PNA can also be used. The nucleotide units in each probe can be either naturally occurring residues (such as deoxyadenylate, deoxycytidylate, deoxyguanylate, deoxythymidylate, adenylate, cytidylate, guanylate, and uridylate), or synthetically produced analogs that are capable of forming desired base-pair relationships, or a combination thereof. Examples of these analogs include, but are not limited to, aza and deaza pyrimidine analogs, aza and deaza purine analogs, and other heterocyclic base analogs, wherein one or more of the carbon and nitrogen atoms of the purine and pyrimidine rings are substituted by heteroatoms, such as oxygen, sulfur, selenium, and phosphorus. Similarly, the polynucleotide backbones of the probes can be either naturally occurring (such as through 5′ to 3′ linkage), or modified. For instance, the nucleotide units can be connected via non-typical linkage, such as 5′ to 2′ linkage, so long as the linkage does not interfere with hybridization. For another instance, peptide nucleic acids, in which the constitute bases are joined by peptide bonds rather than phosphodiester linkages, can be used.

In one embodiment, the polynucleotide probes employed in the present invention have relatively high sequence complexity, and do not contain long stretches of the same nucleotide. In another embodiment, the polynucleotide probes employed in the present invention are designed such that they do not have a high proportion of G or C residues at the 3′ ends. In yet another embodiment, the probes do not have a 3′ terminal T residue. Depending on the type of assay or detection to be performed, sequences that are predicted to form hairpins or interstrand structures, such as “primer dimers,” can be either included in, or excluded from, the probe sequences. In still another embodiment, each probe does not contain any ambiguous base.

Any part of a parent sequence can be used to prepare probes. For instance, probes can be prepared from the protein-coding region, the 5′ untranslated region, or the 3′ untranslated region of a parent sequence. Multiple probes, such as at least 5, 10, 15, 20, 25, 30, 40, 50, 60, 70, 80, 90, 100, or more, can be prepared for a given parent sequence. These multiple probes may or may not overlap each other, although overlap among different probes may be desirable in some assays.

In another embodiment, the probes for a parent sequence have low sequence identities with other parent sequences, or the complements thereof. For instance, each probe for a parent sequence can have no more than 70%, 60%, 50% or less sequence identity with other parent sequences, or the complements thereof. This reduces the risk of cross-hybridization between the probes and the undesired RNA transcripts. Sequence identity can be determined using any method known in the art. These methods include, but are not limited to, BLASTN, FASTA, FASTDB, and the GCG program.

The suitability of a probe for hybridization can be evaluated using various computer programs. Programs suitable for this purpose include, but are not limited to, LaserGene (DNAStar), Oligo (National Biosciences, Inc.), MacVector (Kodak/IBI), and the standard programs provided by the Genetics Computer Group (GCG).

The polynucleotide probes of the present invention can be synthesized using any method known in the art. Exemplary methods include automated or high throughput DNA synthesizers, such as those provided by Millipore, GeneMachines, and BioAutomation. In one embodiment, the synthesized probes are substantially free of impurities, such as incomplete products produced during the synthesis. In another embodiment, the probes are substantially free of other contaminants that may hinder the desired functions of the probes. The probes can be purified or concentrated using different methods, such as reverse phase chromatography, ethanol precipitation, gel filtration, electrophoresis, or a combination thereof.

In still another embodiment, the parent sequences with large sizes are divided into shorter sequence segments to facilitate the probe design. These divided sequences, together with the undivided parent sequences, are collectively referred to as the “tiling sequences” (SEQ ID NOs: 4,273-544).

Attachment C depicts the tiling sequences and their corresponding headers. Each header includes a qualifier (after “wyeHumanDT1a”) and other information of the corresponding tiling sequence. Attachment D shows the location of each tiling sequence in the corresponding parent sequence. The 5′ end of each tiling sequence in the corresponding parent sequence is indicated under “TilingStart,” and the 3′ end under “TilingEnd.”

Polynucleotide probes for each tiling sequence can hybridize under stringent or nucleic acid array hybridization conditions to that tiling sequence, or the complement thereof. In one embodiment, a probe for a tiling sequence can hybridize under highly stringent conditions to the tiling sequence, or the complement thereof. In another embodiment, the probes for a tiling sequence are incapable of hybridizing under stringent or nucleic acid array hybridization conditions to other tiling sequences, or the complements thereof. If a tiling sequence contains one or more ambiguous residues, the probes for that tiling sequence can prepared such that they are capable of hybridizing under stringent or nucleic acid array hybridization conditions to an unambiguous segment of the tiling sequence, or the complement of the unambiguous segment.

Any method known in the art may be used to prepare probes for the tiling sequences. In one embodiment, the probes are generated using Array Designer, a software package provided by TeleChem International, Inc (Sunnyvale, Calif. 94089). Examples of the probes thus generated are illustrated in Attachment F (SEQ ID NOs: 8,647-116,337). The locations of the 5′ and 3′ ends of each probe in the corresponding tiling sequence are shown under “5′ End” and “3′ End,” respectively. The qualifier of each probe, which indicative the corresponding tiling sequence from which the probe is derived, is also indicated. Other methods or software programs can also be used to generate hybridization probes for the tiling sequences of the present invention.

The parent sequences, tiling sequences, and polynucleotide probes of the present invention can be used to detect or monitor the expression profiles of human drug target genes. Methods suitable for this purpose include, but are not limited to, nucleic acid arrays (including bead arrays), Southern Blot, Northern Blot, in situ hybridization, PCR, and RT-PCR.

C. Nucleic Acid Arrays for Detecting Expression Profiles of Human Drug Target Genes

The polynucleotide probes of the present invention can be used to make nucleic acid arrays. A typical nucleic acid array includes at least one substrate support which includes a plurality of discrete regions. The location of each discrete region is either known or determinable. These discrete regions can be organized in various forms or patterns. For instance, the discrete regions can be arranged as an array of regularly spaced areas on the surface of a substrate. Other patterns, such as linear, concentric or spiral patterns, can also be used. In one embodiment, a nucleic acid array of the present invention is a bead array which includes a plurality of beads stably associated with the polynucleotide probes of the present invention.

Polynucleotide probes can be stably attached to the corresponding discrete regions through covalent or non-covalent interactions. By “stably attached” or “stably associated,” it means that during nucleic acid array hybridization the polynucleotide probe maintains its position relative to the discrete region to which the probe is attached. Any suitable method can be used to attach polynucleotide probes to a nucleic acid array substrate. In one embodiment, the attachment is achieved by first depositing the polynucleotide probes to their respective discrete regions and then exposing the surface to a solution of a cross-linking agent, such as glutaraldehyde, borohydride, or other bifunctional agents. In another embodiment, the polynucleotide probes are covalently bound to the substrate via an alkylamino-linker group or by coating the glass slides with polyethylenimine followed by activation with cyanuric chloride for coupling the polynucleotides. In yet another embodiment, the polynucleotide probes are covalently attached to the nucleic acid array through polymer linkers. The polymer linkers may improve the accessibility of the probes to their purported targets. In many cases, the polymer linkers are not involved in the interactions between the probes and their purported targets.

In addition, polynucleotide probes can be stably attached to a nucleic acid array substrate through non-covalent interactions. In one embodiment, polynucleotide probes are attached to a substrate through electrostatic interactions between positively charged surface groups and negatively charged probes. In another embodiment, the substrate is a glass slide having a coating of a polycationic polymer on its surface, such as a cationic polypeptide. The probes are bound to these polycationic polymers. In yet another embodiment, the methods described in U.S. Pat. No. 6,440,723 are used to attach polynucleotide probes to a nucleic acid array substrate.

Various materials can be used to make the substrate supports of nucleic acid arrays. Suitable materials include, but are not limited to, glasses, silica, ceramics, nylons, quartz wafers, gels, metals, and papers. The substrates can be flexible or rigid. In one embodiment, they are in the form of a tape that is wound up on a reel or cassette. Two or more substrate supports can be used in the same nucleic acid array. In many cases, the substrates are not reactive with reagents that are used in nucleic acid array hybridization.

The surface(s) of a substrate support can be smooth and substantially planar. The surface(s) of a substrate can also have a variety of configurations, such as raised or depressed regions, trenches, v-grooves, mesa structures, or other regularities or irregularities. The surface(s) of a substrate can be coated with one or more modification layers. Suitable modification layers include inorganic and organic layers, such as metals, metal oxides, polymers, or small organic molecules. In one embodiment, the surface(s) of a substrate is chemically treated to include groups such as hydroxyl, carboxyl, amine, aldehyde, or sulfhydryl groups.

The discrete regions on a substrate can be of any size, shape and density. For instance, they can be squares, ellipsoids, rectangles, triangles, circles, other regular or irregular geometric shapes, or any portion or combination thereof. In one embodiment, each discrete region has a surface area of less than 10⁻¹ cm², such as less than 10⁻², 10⁻³, 10⁻⁴, 10⁻⁵, 10⁻⁶, or 10⁻⁷ cm². In another embodiment, the spacing between each discrete region and its closest neighbor, measured from center-to-center, is in the range of from about 10 to about 400 μm. The density of the discrete regions may range, for example, between 50 and 50,000 regions/cm².

All of the methods known in the art can be used to make the nucleic acid arrays of the present invention. For instance, the probes can be synthesized in a step-by-step manner on a substrate, or can be attached to a substrate in pre-synthesized forms. Algorithms for reducing the number of synthesis cycles can be used. In one embodiment, a nucleic acid array of the present invention is synthesized in a combinational fashion by delivering monomers to the discrete regions through mechanically constrained flowpaths. In another embodiment, a nucleic acid array of the present invention is synthesized by spotting monomer reagents onto a substrate support using an ink jet printer (such as the DeskWriter C manufactured by Hewlett-Packard). In yet another embodiment, polynucleotide probes are immobilized on a nucleic acid array using photolithography techniques.

The nucleic acid arrays of the present invention can also be bead arrays which include a plurality of beads. Polynucleotide probes can be stably attached to these beads using the methods described above.

In one embodiment, a substantial portion of all of the polynucleotide probes that are stably attached to a nucleic acid array of the present invention is probes for drug target genes. For instance, at least 15%, 20%, 25%, 30%, 35%, 40%, 45%, 50%, or more of all of the polynucleotide probes that are stably attached to the nucleic acid array are drug target gene probes. In one example, all of these drug target gene probes are attached to one substrate support. In another example, these drug target gene probes are attached to two or more substrate supports. Examples of drug target genes include, but are not limited to, kinase genes, phosphatase genes, protease genes, G-protein coupled receptor genes, nuclear hormone receptor genes, and ion channel genes.

Any number of polynucleotide probes can be included in a nucleic acid array of the present invention. In one embodiment, a nucleic acid array of the present invention includes at least 2, 5, 10, 20, 30, 40, 50, 100, 200, 300, 400, 500, 1,000, or more different probes, and each probe can hybridize under stringent or nucleic acid array hybridization conditions to a different respective drug target gene. In another embodiment, a nucleic acid array of the present invention includes at least 2, 5, 10, 50, 100, 500, or more probes for each class of drug target genes.

In still another embodiment, a nucleic acid array of the present invention includes at least 2, 5, 10, 20, 30, 40, 50, 100, 200, 300, 400, 500, 1,000, 2,000, 3,000, 4,000, or more different probes, and each probe can hybridize under stringent or nucleic acid array hybridization conditions to a different respective tiling sequence selected from Attachment C, or the full complement thereof. In another embodiment, a nucleic acid array of the present invention comprises at least one probe for each tiling sequence selected from Attachment C.

In yet another embodiment, a nucleic acid array of the present invention includes at least 1, 2, 5, 10, 20, 30, 40, 50, or more probes for an ADAMTS4 gene. In one example, the ADAMTS4 probes can hybridize under stringent or nucleic acid array hybridization conditions to a sequence selected from the group consisting of SEQ ID NO: 6,797 (tiling: wyeHumanDT1a:NM_{—}005099.2_at), SEQ ID NO: 6,798 (tiling:wyeHumanDT1a:NM_{—}005099.2_s_at), and the full complements thereof. In another example, the ADAMTS4 probes are selected from Attachment F (e.g., SEQ ID NOs: 46,292-46,335).

Multiple probes can be included in a nucleic acid array to detect the same drug target gene or tiling sequence. For instance, at least 2, 5, 10, 15, 20, 25, 30, 35, 40, 45, 50, 55, 60, 65, 70, or more probes can be used to detect the same drug target gene or tiling sequence. In one example, the nucleic acid array includes at least from 35 to 68 probes for each drug target gene or tiling sequence of interest. Reliability and reproducibility of probe set signal values decrease substantially if less than 20 probe pair per transcript are used. By increasing the number of probe pairs for each drug target gene or tile sequence, a more robust and reliable detection can be achieved.

Each probe can be attached to a different respective discrete region on the nucleic acid array. Alternatively, two or more different probes can be attached to the same discrete region. The concentration of one probe with respect to the other probe or probes in the same region may vary according to the objectives and requirements of the particular experiment. In one embodiment, different probes in the same region are present in approximately equivocal ratio.

In many instances, probes for different tiling sequences are attached to different discrete regions on a nucleic acid array. In some embodiments, however, probes for different tiling sequences are attached to the same discrete region.

As discussed above, the length of each probe on a nucleic acid array can be selected to achieve the desired hybridization effects. For instance, each probe can include or consist of 15, 20, 25, 30, 35, 40, 45, 50, 60, 70, 80, 90, 100 or more consecutive nucleotides. In one embodiment, each probe consists of 25 consecutive nucleotides. In another embodiment, a nucleic acid array includes each and every oligonucleotide probe selected from Attachment F, or the full complement thereof.

The nucleic acid arrays of the present invention can further include control probes which can hybridize under stringent or nucleic acid array hybridization conditions to corresponding control sequences, or the complements thereof. Exemplary control sequences are depicted in SEQ ID Nos: 8545-8646. Table 2 shows the header information for each control sequence. Each header includes aqualifier (after “wyeHumanDT1a”), as well as other information, of the corresponding control sequence.

TABLE 2
Control Sequences

| SEQ | |
|---|---|
| ID NO | Header |
| 8545 | “>control: wyeHumanDT1a: AFFX-BioB-5_at; J04423; E coli bioB gene |
| | biotin synthetase (-5, -M, -3 represent transcript regions 5 prime, Middle, |
| | and 3 prime respectively)” |
| 8546 | “>control: wyeHumanDT1a: AFFX-BioB-M_at; J04423; E coli bioB gene |
| | biotin synthetase (-5, -M, -3 represent transcript regions 5 prime, Middle, |
| | and 3 prime respectively)” |
| 8547 | “>control: wyeHumanDT1a: AFFX-BioB-3_at; J04423; E coli bioB gene |
| | biotin synthetase (-5, -M, -3 represent transcript regions 5 prime, Middle, |
| | and 3 prime respectively)” |
| 8548 | >control: wyeHumanDT1a: AFFX-BioC-5_at; J04423; E coli bioC protein (-5 |
| | and -3 represent transcript regions 5 prime and 3 prime respectively) |
| 8549 | >control: wyeHumanDT1a: AFFX-BioC-3_at; J04423; E coli bioC protein (-5 |
| | and -3 represent transcript regions 5 prime and 3 prime respectively) |
| 8550 | >control: wyeHumanDT1a: AFFX-BioDn-5_at; J04423; E coli bioD gene |
| | dethiobiotin synthetase (-5 and -3 represent transcript regions 5 prime and 3 |
| | prime respectively) |
| 8551 | >control: wyeHumanDT1a: AFFX-BioDn-3_at; J04423; E coli bioD gene |
| | dethiobiotin synthetase (-5 and -3 represent transcript regions 5 prime and 3 |
| | prime respectively) |
| 8552 | >control: wyeHumanDT1a: AFFX-CreX-5_at; X03453; Bacteriophage P1 |
| | cre recombinase protein (-5 and -3 represent transcript regions 5 prime and |
| | 3 prime respectively) |
| 8553 | >control: wyeHumanDT1a: AFFX-CreX-3_at; X03453; Bacteriophage P1 |
| | cre recombinase protein (-5 and -3 represent transcript regions 5 prime and |
| | 3 prime respectively) |
| 8554 | “>control: wyeHumanDT1a: AFFX-DapX-5_at; L38424; B subtilis dapB, |
| | jojF, jojG genes corresponding to nucleotides 1358-3197 of L38424 (-5, -M, |
| | -3 represent transcript regions 5 prime, Middle, and 3 prime respectively)” |
| 8555 | “>control: wyeHumanDT1a: AFFX-DapX-M_at; L38424; B subtilis dapB, |
| | jojF, jojG genes corresponding to nucleotides 1358-3197 of L38424 (-5, -M, |
| | -3 represent transcript regions 5 prime, Middle, and 3 prime respectively)” |
| 8556 | “>control: wyeHumanDT1a: AFFX-DapX-3_at; L38424; B subtilis dapB, |
| | jojF, jojG genes corresponding to nucleotides 1358-3197 of L38424 (-5, -M, |
| | -3 represent transcript regions 5 prime, Middle, and 3 prime respectively)” |
| 8557 | “>control: wyeHumanDT1a: AFFX-LysX-5_at; X17013; B subtilis lys gene |
| | for diaminopimelate decarboxylase corresponding to nucleotides 350-1345 |
| | of X17013 (-5, -M, -3 represent transcript regions 5 prime, Middle, and 3 |
| | prime respectively)” |
| 8558 | “>control: wyeHumanDT1a: AFFX-LysX-M at; X17013; B subtilis lys gene |
| | for diaminopimelate decarboxylase corresponding to nucleotides 350-1345 |
| | of X17013 (-5, -M, -3 represent transcript regions 5 prime, Middle, and 3 |
| | prime respectively)” |
| 8559 | “>control: wyeHumanDT1a: AFFX-LysX-3_at; X17013; B subtilis lys gene |
| | for diaminopimelate decarboxylase corresponding to nucleotides 350-1345 |
| | of X17013 (-5, -M, -3 represent transcript regions 5 prime, Middle, and 3 |
| | prime respectively)” |
| 8560 | “>control: wyeHumanDT1a: AFFX-PheX-5_at; M24537; B subtilis pheB, |
| | pheA genes corresponding to nucleotides 2017-3334 of M24537 (-5, -M, -3 |
| | represent transcript regions 5 prime, Middle, and 3 prime respectively)” |
| 8561 | “>control: wyeHumanDT1a: AFFX-PheX-M_at; M24537; B subtilis pheB, |
| | pheA genes corresponding to nucleotides 2017-3334 of M24537 (-5, -M, -3 |
| | represent transcript regions 5 prime, Middle, and 3 prime respectively)” |
| 8562 | “>control: wyeHumanDT1a: AFFX-PheX-3_at; M24537; B subtilis pheB, |
| | pheA genes corresponding to nucleotides 2017-3334 of M24537 (-5, -M, -3 |
| | represent transcript regions 5 prime, Middle, and 3 prime respectively)” |
| 8563 | “>control: wyeHumanDT1a: AFFX-ThrX-5_at; X04603; B subtilis thrC, |
| | thrB genes corresponding to nucleotides 248-2229 of X04603 (-5, -M, -3 |
| | represent transcript regions 5 prime, Middle, and 3 prime respectively)” |
| 8564 | “>control: wyeHumanDT1a: AFFX-ThrX-M_at; X04603; B subtilis thrC, |
| | thrB genes corresponding to nucleotides 248-2229 of X04603 (-5, -M, -3 |
| | represent transcript regions 5 prime, Middle, and 3 prime respectively)” |
| 8565 | “>control: wyeHumanDT1a: AFFX-ThrX-3_at; X04603; B subtilis thrC, |
| | thrB genes corresponding to nucleotides 248-2229 of X04603 (-5, -M, -3 |
| | represent transcript regions 5 prime, Middle, and 3 prime respectively)” |
| 8566 | “>control: wyeHumanDT1a: AFFX-TrpnX-5_at; K01391; B subtilis TrpE |
| | protein, TrpD protein, TrpC protein corresponding to nucleotides 1883-4400 |
| | of K01391 (-5, -M, -3 represent transcript regions 5 prime, Middle, and 3 |
| | prime respectively)” |
| 8567 | “>control: wyeHumanDT1a: AFFX-TrpnX-M_at; K01391; B subtilis TrpE |
| | protein, TrpD protein, TrpC protein corresponding to nucleotides 1883-4400 |
| | of K01391 (-5, -M, -3 represent transcript regions 5 prime, Middle, and 3 |
| | prime respectively)” |
| 8568 | “>control: wyeHumanDT1a: AFFX-TrpnX-3_at; K01391; B subtilis TrpE |
| | protein, TrpD protein, TrpC protein corresponding to nucleotides 1883-4400 |
| | of K01391 (-5, -M, -3 represent transcript regions 5 prime, Middle, and 3 |
| | prime respectively)” |
| 8569 | “>control: wyeHumanDT1a: AFFX-r2-Ec-bioB-5_at; J04423; Escherichia |
| | coli /REF = J04423 /DEF = E coli bioB gene biotin synthetase corresponding |
| | to nucleotides 2071-2304 of J04423 /LEN = 1114 (-5, -M, -3 represent |
| | transcript regions 5 prime, Middle, and 3 prime respectively)” |
| 8570 | “>control: wyeHumanDT1a: AFFX-r2-Ec-bioB-M_at; J04423; Escherichia |
| | coli /REF = J04423 /DEF = E coli bioB gene biotin synthetase corresponding |
| | to nucleotides 2393-2682 of J04423 /LEN = 1114 (-5, -M, -3 represent |
| | transcript regions 5 prime, Middle, and 3 prime respectively)” |
| 8571 | “>control: wyeHumanDT1a: AFFX-r2-Ec-bioB-3_at; J04423; Escherichia |
| | coli /REF = J04423 /DEF = E coli bioB gene biotin synthetase corresponding |
| | to nucleotides 2772-3004 of J04423 /LEN = 1114 (-5, -M, -3 represent |
| | transcript regions 5 prime, Middle, and 3 prime respectively)” |
| 8572 | >control: wyeHumanDT1a: AFFX-r2-Ec-bioC-5_at; J04423; Escherichia coli |
| | /REF = J04423 /DEF = E coli bioC protein corresponding to nucleotides 4257- |
| | 4573 of J04423 /LEN = 777 (-5 and -3 represent transcript regions 5 prime |
| | and 3 prime respectively) |
| 8573 | >control: wyeHumanDT1a: AFFX-r2-Ec-bioC-3_at; J04423; Escherichia coli |
| | /REF = J04423 /DEF = E coli bioC protein corresponding to nucleotides 4609- |
| | 4883 of J04423 /LEN = 777 (-5 and -3 represent transcript regions 5 prime |
| | and 3 prime respectively) |
| 8574 | >control: wyeHumanDT1a: AFFX-r2-Ec-bioD-5_at; J04423; Escherichia |
| | coli /REF = J04423 /DEF = E coli bioD gene dethiobiotin synthetase |
| | corresponding to nucleotides 5024-5244 of J04423 /LEN = 676 (-5 and -3 |
| | represent transcript regions 5 prime and 3 prime respectively) |
| 8575 | >control: wyeHumanDT1a: AFFX-r2-Ec-bioD-3_at; J04423; Escherichia |
| | coli /REF = J04423 /DEF = E coli bioD gene dethiobiotin synthetase |
| | corresponding to nucleotides 5312-5559 of J04423 /LEN = 676 (-5 and -3 |
| | represent transcript regions 5 prime and 3 prime respectively) |
| 8576 | >control: wyeHumanDT1a: AFFX-r2-P1-cre-5_at; X03453; Bacteriophage |
| | /REF = X03453 /DEF = Bacteriophage P1 cre recombinase protein |
| | corresponding to nucleotides 581-1001 of X03453 /LEN = 1058 (-5 and -3 |
| | represent transcript regions 5 prime and 3 prime respectively) |
| 8577 | >control: wyeHumanDT1a: AFFX-r2-P1-cre-3_at; X03453; Bacteriophage |
| | /REF = X03453 /DEF = Bacteriophage P1 cre recombinase protein |
| | corresponding to nucleotides 1032-1270 of X03453 /LEN = 1058 (-5 and -3 |
| | represent transcript regions 5 prime and 3 prime respectively) |
| 8578 | “>control: wyeHumanDT1a: AFFX-r2-Bs-dap-5_at; L38424; Bacillus |
| | subtilis /REF = L38424 /DEF = B subtilis dapB, jojF, jojG genes |
| | corresponding to nucleotides 1439-1846 of L38424 /LEN = 1931 (-5, -M, -3 |
| | represent transcript regions 5 prime, Middle, and 3 prime respectively)” |
| 8579 | “>control: wyeHumanDT1a: AFFX-r2-Bs-dap-M_at; L38424; Bacillus |
| | subtilis /REF = L38424 /DEF = B subtilis dapB, jojF, jojG genes |
| | corresponding to nucleotides 2055-2578 of L38424 /LEN = 1931 (-5, -M, -3 |
| | represent transcript regions 5 prime, Middle, and 3 prime respectively)” |
| 8580 | “>control: wyeHumanDT1a: AFFX-r2-Bs-dap-3_at; L38424; Bacillus |
| | subtilis /REF = L38424 /DEF = B subtilis dapB, jojF, jojG genes |
| | corresponding to nucleotides 2634-3089 of L38424 /LEN = 1931 (-5, -M, -3 |
| | represent transcript regions 5 prime, Middle, and 3 prime respectively)” |
| 8581 | “>control: wyeHumanDT1a: AFFX-r2-Bs-lys-5_at; X17013; Bacillus subtilis |
| | /REF = X17013 /DEF = B subtilis lys gene for diaminopimelate decarboxylase |
| | corresponding to nucleotides 411-659 of X17013 /LEN = 1108 (-5, -M, -3 |
| | represent transcript regions 5 prime, Middle, and 3 prime respectively)” |
| 8582 | “>control: wyeHumanDT1a: AFFX-r2-Bs-lys-M_at; X17013; Bacillus |
| | subtilis /REF = X17013 /DEF = B subtilis lys gene for diaminopimelate |
| | decarboxylase corresponding to nucleotides 673-1002 of X17013 |
| | /LEN = 1108 (-5, -M, -3 represent transcript regions 5 prime, Middle, and 3 |
| | prime respectively)” |
| 8583 | “>control: wyeHumanDT1a: AFFX-r2-Bs-lys-3_at; X17013; Bacillus subtilis |
| | /REF = X17013 /DEF = B subtilis lys gene for diaminopimelate decarboxylase |
| | corresponding to nucleotides 1008-1263 of X17013 /LEN = 1108 (-5, -M, -3 |
| | represent transcript regions 5 prime, Middle, and 3 prime respectively)” |
| 8584 | “>control: wyeHumanDT1a: AFFX-r2-Bs-phe-5_at; M24537; Bacillus |
| | subtilis /REF = M24537 /DEF = B subtilis pheB, pheA genes corresponding to |
| | nucleotides 2116-2382 of M24537 /LEN = 1409 (-5, -M, -3 represent |
| | transcript regions 5 prime, Middle, and 3 prime respectively)” |
| 8585 | “>control: wyeHumanDT1a: AFFX-r2-Bs-phe-M_at; M24537; Bacillus |
| | subtilis /REF = M24537 /DEF = B subtilis pheB, pheA genes corresponding to |
| | nucleotides 2484-2875 of M24537 /LEN = 1409 (-5, -M, -3 represent |
| | transcript regions 5 prime, Middle, and 3 prime respectively)” |
| 8586 | “>control: wyeHumanDT1a: AFFX-r2-Bs-phe-3_at; M24537; Bacillus |
| | subtilis /REF = M24537 /DEF = B subtilis pheB, pheA genes corresponding to |
| | nucleotides 2897-3200 of M24537 /LEN = 1409 (-5, -M, -3 represent |
| | transcript regions 5 prime, Middle, and 3 prime respectively)” |
| 8587 | “>control: wyeHumanDT1a: AFFX-r2-Bs-thr-5_s_at; X04603; Bacillus |
| | subtilis /REF = X04603 /DEF = B subtilis thrC, thrB genes corresponding to |
| | nucleotides 288-932 of X04603 /LEN = 2073 (-5, -M, -3 represent transcript |
| | regions 5 prime, Middle, and 3 prime respectively)” |
| 8588 | “>control: wyeHumanDT1a: AFFX-r2-Bs-thr-M_s_at; X04603; Bacillus |
| | subtilis /REF = X04603 /DEF = B subtilis thrC, thrB genes corresponding to |
| | nucleotides 995-1562 of X04603 /LEN = 2073 (-5, -M, -3 represent transcript |
| | regions 5 prime, Middle, and 3 prime respectively)” |
| 8589 | “>control: wyeHumanDT1a: AFFX-r2-Bs-thr-3_s_at; X04603; Bacillus |
| | subtilis /REF = X04603 /DEF = B subtilis thrC, thrB genes corresponding to |
| | nucleotides 1689-2151 of X04603 /LEN = 2073 (-5, -M, -3 represent |
| | transcript regions 5 prime, Middle, and 3 prime respectively)” |
| 8590 | >control: wyeHumanDT1a: AFFX-HSAC07/X00351_5_at; X00351; Human |
| | mRNA for beta-actin. |
| 8591 | >control: wyeHumanDT1a: AFFX-HSAC07/X00351_M_at; X00351; |
| | Human mRNA for beta-actin. |
| 8592 | >control: wyeHumanDT1a: AFFX-HSAC07/X00351_3_at; X00351; Human |
| | mRNA for beta-actin. |
| 8593 | >control: wyeHumanDT1a: AFFX-hum_alu_at; U14573; ***ALU |
| | WARNING: Human Alu-Sq subfamily consensus sequence. |
| 8594 | “>control: wyeHumanDT1a: AFFX-HUMGAPDH/M33197_5_at; M33197; |
| | Human glyceraldehyde-3-phosphate dehydrogenase (GAPDH) mRNA, |
| | complete cds.” |
| 8595 | “>control: wyeHumanDT1a: AFFX-HUMGAPDH/M33197_M_at; M33197; |
| | Human glyceraldehyde-3-phosphate dehydrogenase (GAPDH) mRNA, |
| | complete cds.” |
| 8596 | “>control: wyeHumanDT1a: AFFX-HUMGAPDH/M33197_3_at; M33197; |
| | Human glyceraldehyde-3-phosphate dehydrogenase (GAPDH) mRNA, |
| | complete cds.” |
| 8597 | “>control: wyeHumanDT1a: AFFX-HUMISGF3A/M97935_5_at; M97935; |
| | Homo sapiens transcription factor ISGF-3 mRNA, complete cds.” |
| 8598 | “>control: wyeHumanDT1a: AFFX-HUMISGF3A/M97935_MA_at; |
| | M97935; Homo sapiens transcription factor ISGF-3 mRNA, complete cds.” |
| 8599 | “>control: wyeHumanDT1a: AFFX-HUMISGF3A/M97935_MB_at; |
| | M97935; Homo sapiens transcription factor ISGF-3 mRNA, complete cds.” |
| 8600 | “>control: wyeHumanDT1a: AFFX-HUMISGF3A/M97935_3_at; M97935; |
| | Homo sapiens transcription factor ISGF-3 mRNA, complete cds.” |
| 8601 | “>control: wyeHumanDT1a: AFFX-HUMRGE/M10098_5_at; M10098; |
| | Human 18S rRNA gene, complete.” |
| 8602 | “>control: wyeHumanDT1a: AFFX-HUMRGE/M10098_M_at; M10098; |
| | Human 18S rRNA gene, complete.” |
| 8603 | “>control: wyeHumanDT1a: AFFX-HUMRGE/M10098_3_at; M10098; |
| | Human 18S rRNA gene, complete.” |
| 8604 | “>control: wyeHumanDT1a: AFFX-M27830_5_at; M27830; Human 28S |
| | ribosomal RNA gene, complete cds.” |
| 8605 | “>control: wyeHumanDT1a: AFFX-M27830_M_at; M27830; Human 28S |
| | ribosomal RNA gene, complete cds.” |
| 8606 | “>control: wyeHumanDT1a: AFFX-M27830_3_at; M27830; Human 28S |
| | ribosomal RNA gene, complete cds.” |
| 8607 | “>control: wyeHumanDT1a: BIOB5_at; J04423; E. coli 7,8-diamino- |
| | pelargonic acid (bioA), biotin synthetase (bioB), 7-keto-8-amino-pelargonic |
| | acid synthetase (bioF), bioC protein, and dethiobiotin synthetase (bioD), |
| | complete cds.” |
| 8608 | “>control: wyeHumanDT1a: BIOBM_at; J04423; E. coli 7,8-diamino- |
| | pelargonic acid (bioA), biotin synthetase (bioB), 7-keto-8-amino-pelargonic |
| | acid synthetase (bioF), bioC protein, and dethiobiotin synthetase (bioD), |
| | complete cds.” |
| 8609 | “>control: wyeHumanDT1a: BIOB3_at; J04423; E. coli 7,8-diamino- |
| | pelargonic acid (bioA), biotin synthetase (bioB), 7-keto-8-amino-pelargonic |
| | acid synthetase (bioF), bioC protein, and dethiobiotin synthetase (bioD), |
| | complete cds.” |
| 8610 | “>control: wyeHumanDT1a: BIOC5_at; J04423; E. coli 7,8-diamino- |
| | pelargonic acid (bioA), biotin synthetase (bioB), 7-keto-8-amino-pelargonic |
| | acid synthetase (bioF), bioC protein, and dethiobiotin synthetase (bioD), |
| | complete cds.” |
| 8611 | “>control: wyeHumanDT1a: BIOC3_at; J04423; E. coli 7,8-diamino- |
| | pelargonic acid (bioA), biotin synthetase (bioB), 7-keto-8-amino-pelargonic |
| | acid synthetase (bioF), bioC protein, and dethiobiotin synthetase (bioD), |
| | complete cds.” |
| 8612 | “>control: wyeHumanDT1a: BIOD5_at; J04423; E. coli 7,8-diamino- |
| | pelargonic acid (bioA), biotin synthetase (bioB), 7-keto-8-amino-pelargonic |
| | acid synthetase (bioF), bioC protein, and dethiobiotin synthetase (bioD), |
| | complete cds.” |
| 8613 | “>control: wyeHumanDT1a: BIOD3_at; J04423; E. coli 7,8-diamino- |
| | pelargonic acid (bioA), biotin synthetase (bioB), 7-keto-8-amino-pelargonic |
| | acid synthetase (bioF), bioC protein, and dethiobiotin synthetase (bioD), |
| | complete cds.” |
| 8614 | >control: wyeHumanDT1a: CRE5_at; X03453; Bacteriophage P1 cre gene |
| | for recombinase protein. |
| 8615 | >control: wyeHumanDT1a: CRE3_at; X03453; Bacteriophage P1 cre gene |
| | for recombinase protein. |
| 8616 | “>control: wyeHumanDT1a: DAP5_at; L38424; Bacillus subtilis |
| | dihydropicolinate reductase (jojE) gene, complete cds; poly(A) polymerase |
| | (jojI) gene, complete cds; biotin acetyl-CoA-carboxylase ligase (birA) gene, |
| | complete cds; jojC, jojD, jojF, jojG, jojH genes, complete cds's.” |
| 8617 | “>control: wyeHumanDT1a: DAPM_at; L38424; Bacillus subtilis |
| | dihydropicolinate reductase (jojE) gene, complete cds; poly(A) polymerase |
| | (jojI) gene, complete cds; biotin acetyl-CoA-carboxylase ligase (birA) gene, |
| | complete cds; jojC, jojD, jojF, jojG, jojH genes, complete cds's.” |
| 8618 | “>control: wyeHumanDT1a: DAP3_at; L38424; Bacillus subtilis |
| | dihydropicolinate reductase (jojE) gene, complete cds; poly(A) polymerase |
| | (jojI) gene, complete cds; biotin acetyl-CoA-carboxylase ligase (birA) gene, |
| | complete cds; jojC, jojD, jojF, jojG, jojH genes, complete cds's.” |
| 8619 | >control: wyeHumanDT1a: LYSA5_at; X17013; Bacillus subtilis lys gene |
| | for diaminopimelate decarboxylase (EC 4.1.1.20). |
| 8620 | >control: wyeHumanDT1a: LYSAM_at; X17013; Bacillus subtilis lys gene |
| | for diaminopimelate decarboxylase (EC 4.1.1.20). |
| 8621 | >control: wyeHumanDT1a: LYSA3_at; X17013; Bacillus subtilis lys gene |
| | for diaminopimelate decarboxylase (EC 4.1.1.20). |
| 8622 | “>control: wyeHumanDT1a: PHE5_at; M24537; Bacillus subtillis |
| | sporulation protein (spoOB), GTP-binding protein (obg), phenylalanine |
| | biosynthesis associated protein (pheB), and monofunctional prephenate |
| | dehydratase (pheA) genes, complete cds.” |
| 8623 | “>control: wyeHumanDT1a: PHEM_at; M24537; Bacillus subtillis |
| | sporulation protein (spoOB), GTP-binding protein (obg), phenylalanine |
| | biosynthesis associated protein (pheB), and monofunctional prephenate |
| | dehydratase (pheA) genes, complete cds.” |
| 8624 | “>control: wyeHumanDT1a: PHE3_at; M24537; Bacillus subtillis |
| | sporulation protein (spoOB), GTP-binding protein (obg), phenylalanine |
| | biosynthesis associated protein (pheB), and monofunctional prephenate |
| | dehydratase (pheA) genes, complete cds.” |
| 8625 | “>control: wyeHumanDT1a: THR5_at; X04603; B. subtilis thrB and thrC |
| | genes for homoserine kinase and threonine synthase (EC 2.7.1.39 and EC |
| | 4.2.99.2, respectively).” |
| 8626 | “>control: wyeHumanDT1a: THRM_at; X04603; B. subtilis thrB and thrC |
| | genes for homoserine kinase and threonine synthase (EC 2.7.1.39 and EC |
| | 4.2.99.2, respectively).” |
| 8627 | “>control: wyeHumanDT1a: THR3_at; X04603; B. subtilis thrB and thrC |
| | genes for homoserine kinase and threonine synthase (EC 2.7.1.39 and EC |
| | 4.2.99.2, respectively).” |
| 8628 | “>control: wyeHumanDT1a: TRP5_at; K01391; B. subtilis tryptophan (trp) |
| | operon, complete cds.” |
| 8629 | “>control: wyeHumanDT1a: TRPM_at; K01391; B. subtilis tryptophan (trp) |
| | operon, complete cds.” |
| 8630 | “>control: wyeHumanDT1a: TRP3_at; K01391; B. subtilis tryptophan (trp) |
| | operon, complete cds.” |
| 8631 | “>control: wyeHumanDT1a: 18SRNA5_Hs_at; M10098; Human 18S rRNA |
| | gene, complete.” |
| 8632 | “>control: wyeHumanDT1a: 18SRNAM_Hs_at; M10098; Human 18S rRNA |
| | gene, complete.” |
| 8633 | “>control: wyeHumanDT1a: 18SRNA3_Hs_at; M10098; Human 18S rRNA |
| | gene, complete.” |
| 8634 | >control: wyeHumanDT1a: BACTIN5_Hs_at; X00351; Human mRNA for |
| | beta-actin. |
| 8635 | >control: wyeHumanDT1a: BACTINM_Hs_at; X00351; Human mRNA for |
| | beta-actin. |
| 8636 | >control: wyeHumanDT1a: BACTIN3_Hs_at; X00351; Human mRNA for |
| | beta-actin. |
| 8637 | “>control: wyeHumanDT1a: GAPDH5_Hs_at; M33197; Human |
| | glyceraldehyde-3-phosphate dehydrogenase (GAPDH) mRNA, complete cds.” |
| 8638 | “>control: wyeHumanDT1a: GAPDHM_Hs_at; M33197; Human |
| | glyceraldehyde-3-phosphate dehydrogenase (GAPDH) mRNA, complete cds.” |
| 8639 | “>control: wyeHumanDT1a: GAPDH3_Hs_at; M33197; Human |
| | glyceraldehyde-3-phosphate dehydrogenase (GAPDH) mRNA, complete cds.” |
| 8640 | “>control: wyeHumanDT1a: PYRCRB5_Hs_at; U04641; Human pyruvate |
| | carboxylase (PC) mRNA, complete cds.” |
| 8641 | “>control: wyeHumanDT1a: PYRCRBMA_Hs_at; U04641; Human pyruvate |
| | carboxylase (PC) mRNA, complete cds.” |
| 8642 | “>control: wyeHumanDT1a: PYRCRBMB_Hs_at; U04641; Human pyruvate |
| | carboxylase (PC) mRNA, complete cds.” |
| 8643 | “>control: wyeHumanDT1a: PYRCRB3_Hs_at; U04641; Human pyruvate |
| | carboxylase (PC) mRNA, complete cds.” |
| 8644 | “>control: wyeHumanDT1a: TRANSFR5_Hs_at; M11507; Human |
| | transferrin receptor mRNA, complete cds.” |
| 8645 | “>control: wyeHumanDT1a: TRANSFRM_Hs_at; M11507; Human |
| | transferrin receptor mRNA, complete cds.” |
| 8646 | “>control: wyeHumanDT1a: TRANSFR3_Hs_at; M11507; Human |
| | transferrin receptor mRNA, complete cds.” |

In many embodiments, the nucleic acid arrays of the present invention include at least one mismatch or perfect mismatch probe for each perfect match probe stably attached to the nucleic acid arrays. A perfect mismatch probe has the same sequence as the corresponding perfect match probe except for a homomeric substitution (A to T, T to A, G to C, and C to G) at or near the center of the perfect mismatch probe. For instance, if the perfect match probe has 2n nucleotide residues, the homomeric substitution in the perfect mismatch probe is either at the n or n+1 position, but not at both positions. If the perfect match probe has 2n+1 nucleotide residues, the homomeric substitution in the perfect mismatch probe is at the n+1 position. The center location of the mismatched residue is more likely to destabilize the duplex formed with the target sequence under hybridization conditions. Each perfect match probe and its corresponding perfect mismatch probe can be stably attached to different discrete regions on a nucleic acid array.

The present invention also features protein arrays for detecting or monitoring expression profiles of drug target genes. Each protein array of the present invention includes probes which can specifically bind to protein products of human drug target genes. In one embodiment, the probes on a protein array of the present invention are antibodies. Many of these antibodies can bind to their corresponding proteins with an affinity constant of at least 10⁴ M⁻¹, 10⁵ M⁻¹, 10⁶ M⁻¹, 10⁷ M⁻¹, or stronger. Suitable antibodies for the present invention include, but are not limited to, polyclonal antibodies, monoclonal antibodies, chimeric antibodies, single chain antibodies, synthetic antibodies, Fab fragments, or fragments produced by a Fab expression library. Other peptides, scaffolds, antibody mimics, high-affinity binders, or protein-binding ligands can also be used to construct the protein arrays of the present invention.

Numerous methods are available for immobilizing antibodies or other probes on a protein array of the present invention. Examples of these methods include, but are not limited to, diffusion (e.g., agarose or polyacrylamide gel), surface absorption (e.g., nitrocellulose or PVDF), covalent binding (e.g., silanes or aldehyde), or non-covalent affinity binding (e.g., biotin-streptavidin). Exemplary methods for protein array fabrication include, but are not limited to, ink-jetting, robotic contact printing, photolithography, or piezoelectric spotting. The method described in MacBeath and Schreiber, SCIENCE, 289: 1760-1763 (2000) can also be used. Suitable substrate supports for a protein array of the present invention include, but are not limited to, glass, membranes, mass spectrometer plates, microtiter wells, silica, or beads.

The protein-coding sequence of a drug target gene can be determined by any method known in the art. In one example, the protein-coding sequence of a drug target gene is obtained based on the sequence annotation provided by NCBI or other sequence databases. In another example, the protein-coding sequence of a drug target gene is extracted from the corresponding parent sequence using an ORF prediction program.

In one embodiment, a substantial portion of all of the probes on a protein array of the present invention consists of drug target gene probes. In another embodiment, a substantial portion of all of the probes on a protein array of the present invention consists of antibodies that specifically recognize the protein products of the parent sequences selected from Attachments A or B. The number, specificity or combination of probes on a protein array of the present invention can be determined using the same method as described above for the manufacture of nucleic acid arrays.

D. Applications

The nucleic acid arrays of the present invention can be used for expression profiling of drug target genes. The nucleic acid arrays of the present invention can also be used for the detection, identification, or evaluation of agents that can modulate the expression profiles or functions of drug target genes. In addition, the nucleic acid arrays of the present invention can be used to assess the specificity or toxicity of a drug or a drug candidate. Furthermore, the nucleic acid arrays of the present invention can be used to analyze drug-drug interactions.

Numerous protocols are available for conducing nucleic acid array analysis. Exemplary protocols include those provided by Affymetrix in connection with the use of its GeneChip arrays. Samples amenable to nucleic acid array hybridization can be prepared from any human cell or tissue. Where a nucleic acid array includes probes for non-human drug target genes, samples can be prepared for cells or tissues of the corresponding non-human species.

The sample for hybridization to a nucleic acid array can be either RNA (e.g., mRNA. or cRNA) or DNA (e.g., cDNA). Various methods are available for isolating RNA from tissues. These methods include, but are not limited to, RNeasy kits (provided by QIAGEN), MasterPure kits (provided by Epicentre Technologies), and TRIZOL (provided by Gibco BRL). The RNA isolation protocols provided by Affymetrix can also be used.

In one embodiment, the isolated RNA is amplified or labeled before being hybridized to a nucleic acid array. Suitable RNA amplification methods include, but are not limited to, reverse transcriptase PCR, isothermal amplification, ligase chain reaction, and Qbeta replicase method. The amplification products can be either cDNA or cRNA. In one embodiment, the isolated mRNA is reverse transcribed to cDNA using a reverse transcriptase and a primer consisting of oligo d(T) and a sequence encoding the phage T7 promoter. The cDNA is single stranded. The second strand of the cDNA can be synthesized using a DNA polymerase, combined with an RNase to break up the DNA/RNA hybrid. After synthesis of the double stranded cDNA, T7 RNA polymerase is added to transcribe cRNA from the second strand of the doubled stranded cDNA. In one embodiment, the originally isolated RNA can be hybridized to a nucleic acid array without amplification.

cDNA, cRNA, or other nucleic acid samples can be labeled with one or more labeling moieties to allow for detection of hybridized polynucleotide complexes. The labeling moieties can include compositions that are detectable by spectroscopic, photochemical, biochemical, bioelectronic, immunochemical, electrical, optical or chemical means. The labeling moieties include radioisotopes, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

Nucleic acid samples can be fragmented before being labeled with detectable moieties. Exemplary methods for fragmentation include, for example, heat or ion-mediated hydrolysis.

Hybridization reactions can be performed in absolute or differential hybridization formats. In the absolute hybridization format, polynucleotides derived from one sample are hybridized to the probes in a nucleic acid array. Signals detected after the formation of hybridization complexes correlate to the polynucleotide levels in the sample. In the differential hybridization format, polynucleotides derived from two samples are labeled with different labeling moieties. A mixture of these differently labeled polynucleotides is added to a nucleic acid array. The nucleic acid array is then examined under conditions in which the emissions from the two different labels are individually detectable. In one embodiment, the fluorophores Cy3 and Cy5 (Amersham Pharmacia Biotech, Piscataway, N.J.) are used as the labeling moieties for the differential hybridization format.

Signals gathered from the nucleic acid arrays can be analyzed using commercially available software, such as those provided by Affymetrix or Agilent Technologies. Controls, such as for scan sensitivity, probe labeling and cDNA or cRNA quantitation, can be included in the hybridization experiments. Hybridization signals can be scaled or normalized before being subject to further analysis. For instance, hybridization signals for each individual probe can be normalized to take into account variations in hybridization intensities when more than one array is used under similar test conditions. Hybridization signals can also be normalized using the intensities derived from internal normalization controls contained on each array. In addition, genes with relatively consistent expression levels across the samples can be used to normalize the expression levels of other genes. In one embodiment, probes for certain maintenance genes are included in the nucleic acid array. These genes are chosen because they show stable levels of expression across a diverse set of tissues. Hybridization signals can be normalized or scaled based on the expression levels of these maintenance genes.

In one embodiment, probes for certain exogenous transcripts are included in the nucleic acid array. These transcripts can be chosen such that they show no similarity to eukaryotic transcripts. In one example, eleven exogenous transcripts at different known concentrations are spiked in to each sample. The array is first scaled to a trimmed-mean target value of 100. Based on the scaled hybridization signal of these eleven probe sets, a standard curve can be drawn such that all transcripts present in the sample can be converted from a signal value to a more meaningful concentration value. In another example, a standard curve correlating the signal value read off of the array and known frequency (molarity) can be generated when the array image is read and the probe set expression values are generated. From this standard curve, each signal value can then be converted to a parts per million or picomolarity value. The exogenous controls spiked into each sample can include, for instance, E. coli BioB-5, E. coli BioB-M, E. coli BioB-3, E. coli BioC-5, E. coli BioC-3, E. coli BioD-3, E. coli BioD-5, Bacteriophage P1 Cre-5, Bacteriophage P1 Cre-3, E. coli Dap-5, B. subtilis Dap-M, and B. subtilis Dap-3. These transcripts can be monitored by control probe sets as discussed below.

The control probes can also include probes for human non-drug target genes. These non-drug target genes include, but are not limited to, genes encoding beta-actin, glyceraldehyde-3-phosphate dehydrogenase (GAPDH), transcription factor ISGF-3, 18S rRNA, pyruvate carboxylase (PC), or transferrin receptor.

In one embodiment, the nucleic acid arrays of the present invention are used to detect, identify, or evaluate agents that can modulate the expression profiles of drug target genes. Typically, an agent of interest is first contacted with a cell preparation. mRNA is extracted from the cell preparation and then hybridized to a nucleic acid array of the present invention. Hybridization signals before and after the contact are compared to determine if the agent modulates the expression profile of any drug target gene.

Any type of agents can be evaluated using the present invention. For instance, the agent can be a small molecule, an antibody, a toxin (including a recombinant immunotoxin), a substrate or pseudosubstrate recognizable by a drug target gene product, or a naturally-occurring factor or an analog thereof. Exemplary naturally-occurring factors include, but are not limited to, endocrine factors, paracrine factors, autocrine factors, intracellular factors, and factors interacting with cell receptors. In one embodiment, the agent of interest is an antisense RNA or a double stranded RNA having RNA interference effect (RNAi). Once a lead compound is identified, its derivatives or analogs can be further screened or tested for the optimal modulation effect.

Any in vitro or in vivo assay system can be used in combination of the nucleic acid arrays of the present invention to identify agents capable of modifying the expression profile of drug target genes. Exemplary assay systems include, but are not limited to, in vitro transcription and translation systems, cell lines, primary cell cultures, and tissue cultures. In one embodiment, high-throughput screen methods or compound libraries are employed.

The modulatory effect of an agent in humans or animal models can also be evaluated using the nucleic acid arrays of the present invention. For instance,. an agent can be first administered to a human or animal. A nucleic acid sample is then prepared from the human or animal, and hybridized to a nucleic acid array of the present invention. Hybridization signals are analyzed to determine the effect of the agent on the expression of drug target genes in the human or animal.

An agent identified by the present invention may modulate the expression profile of a drug target gene by any known or unknown mechanism. In one embodiment, the agent can bind to the 5′ untranslated regulatory sequence of the drug target gene, thereby suppressing or enhancing the transcription of the gene. In another embodiment, the agent can modulate the activity of a transcription factor, which in turn controls the expression of the drug target gene. In yet another embodiment, the agent can regulate the degradation, splicing, or other modifications of the RNA transcripts of the drug target gene. In still another embodiment, the agent can affect the expression or function of another protein which is involved in a signal transduction cascade that regulates the drug target gene.

The nucleic acid arrays of the present invention can also be used to evaluate the effect of a compound on the function of a drug target gene. For instance, a drug target gene may be involved in the regulation of the expression of other drug target genes. By monitoring the expression profiles of these downstream drug target genes, the modulatory effect of a compound on the function of the upstream drug target gene can therefore be determined.

In another embodiment, the nucleic acid arrays of the present invention can be used to assess the specificity or toxicity of a drug candidate. An ideal drug candidate modulates only the specified drug target gene(s) without significantly affecting the expression and function of other drug target genes. The nucleic acid arrays of the present invention allow for the identification of compounds that only modulate particular drug target genes but not others. Accordingly, the present invention provides an effective and inexpensive way to conduct the drug specificity/toxicity analysis, thereby accelerating the drug development process.

In yet another embodiment, the nucleic acid arrays of the present invention can be used to investigate drug-drug interactions. Simultaneous administration of several drugs is often necessary to achieve desired therapeutic objectives. For instance, in cancer chemotherapy, antimicrobial therapy or AIDS treatment, drug combination is usually desirable in order to delay the emergence of.drug resistant tumor cells, microbes or viruses. However, drug combination may also cause unexpected adverse effects. These adverse effects can be the results of an unintended activation or suppression of certain signaling pathways. The expression profiles of the components in these signaling pathways can be monitored using the nucleic acid arrays of the present invention, which allows one to determine if a drug combination will produce any unintended effect in these pathways.

The hybridization data generated from the nucleic acid arrays of the present invention can be stored in a database for future analysis. This database can be used as an informational translator that takes information on a gene directly to a compound that has been found to affect the expression of that gene. For instance, if the database reveals that compound X alters the expression of drug target gene Y, and a paper is published reporting that the expression of drug target gene Y is sensitive to a particular signal transduction pathway, then compound X becomes a candidate for modulataing that signal transduction pathway. This effectively leverages the value of the publicly available data on the identification of potential drug candidates.

The agents identified in the present invention can be used to treat patients who have diseases or conditions that are associated with abnormal expression of drug target genes. These agents can be used to correct or reduce the abnormalities in the expression profiles of drug target genes. As used herein, treatment includes therapeutic treatments as well as prophylactic or preventative measures. Those in need of treatment can include individuals already having a particular medical disorder as well as those who may ultimately acquire the disorder (i.e., those needing preventative measures).

The present invention features pharmaceutical compositions comprising the agents identified by the present invention. Each pharmaceutical composition includes an effective amount of an agent that is sufficient to treat the patient or animal in need thereof. The pharmaceutical composition can also include a pharmaceutically acceptable carrier. Non-limiting examples of suitable pharmaceutically acceptable carriers include solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, that are compatible with pharmaceutical administration. The use of these media and carriers for pharmaceutically active substances is well-known in the art.

A pharmaceutical composition of the present invention can be formulated to be compatible with its intended route of administration. Examples of routes of administration include parenteral, intravenous, intradermal, subcutaneous, oral, inhalation, transdermal, rectal, transmucosal, topical, and systemic administration. In one example, the administration is carried out by using an implant.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

A pharmaceutical composition can be administered to a patient in a sufficient dosage such that the active compound in the pharmaceutical composition can modulate the expression profile of a drug target gene of interest. Suitable therapeutic dosages for a compound can range, for example, from 5 mg to 100 mg, from 15 mg to 85 mg, from 30 mg to 70 mg, or from 40 mg to 60 mg. Dosages below 5 mg or above 100 mg can also be used. Compounds can be administered in one dose or multiple doses. The doses can be administered at intervals such as once daily, once weekly, or once monthly. Dosage schedules for administration of a compound can be adjusted based on, for example, the potency of the compound, the half-life of the compound, and the severity of the patient's condition. In one embodiment, the compound is administered as a bolus dose, to maximize its circulating level. In another embodiment, continuous infusions are used after the bolus dose.

Toxicity and therapeutic efficacy of a compound can be determined by standard pharmaceutical procedures in cell culture or experimental animal models. For instance, the LD₅₀ (the dose lethal to 50% of the population) and the ED₅₀ (the dose therapeutically effective in 50% of the population) can be determined. The dose ratio between toxic and therapeutic effects is the therapeutic index, and can be expressed as the ratio LD₅₀/ED₅₀. In many instances, compounds which exhibit large therapeutic indices are selected.

The data obtained from cell culture assays and animal studies can be used in formulating a range of dosages for use in humans. The dosage of such compounds may lie within a range of circulating concentrations that exhibit an ED₅₀ with little or no toxicity. The dosage may vary within this range depending upon the dosage form employed and the route of administration utilized. For any compound used according to the present invention, a therapeutically effective dose can be estimated initially from cell culture assays. A dose may be formulated in animal models to achieve a circulating plasma concentration range that exhibits an IC₅₀ (i.e., the concentration of the test inhibitor which achieves a half-maximal inhibition of symptoms) as determined by cell culture assays. Levels in plasma may be measured, for example, by high performance liquid chromatography. The effects of any particular dosage can be monitored by suitable bioassays. Examples of suitable bioassays include DNA replication assays, transcription-based assays, GDF protein/receptor binding assays, creatine kinase assays, assays based on the differentiation of pre-adipocytes, assays based on glucose uptake in adipocytes, and immunological assays.

The dosage regimen for the administration of composition can be determined by the attending physician based on various factors which modify the action of the compound, the site of pathology, the severity of disease, the patient's age, sex, and diet, the severity of any inflammation, time of administration and other clinical factors. In many instances, systemic or injectable administration is initiated at a dose which is minimally effective, and the dose is increased over a preselected time course until a positive effect is observed. Subsequently, incremental increases in dosage is made limiting to levels that produce a corresponding increase in effect while taking into account any adverse affects that may appear. The addition of other known factors to a final composition may also affect the dosage.

The present invention also contemplates a collection of polynucleotides. In one embodiment, the polynucleotide collection comprises at least 1, 2, 5, 10, 50, 100, 500, 1,000, or more probes capable of hybridizing under stringent or nucleic acid array hybridization conditions to the tiling sequences selected from Attachment C, or the complements thereof. In another embodiment, the polynucleotide collection comprises at least 1, 2, 5, 10, 50, 100, 500, 1,000, or more tiling sequences selected from Attachment C, or the complements thereof. In yet another embodiment, the polynucleotide collection comprises at least 1, 2, 5, 10, 50, 100, 500, 1,000, or more sequences selected from SEQ ID NOs: 1-4,272, or the complements thereof.

Furthermore, the present invention contemplates a collection of probes capable of binding to the protein products encoded by the parent sequences selected from Attachments A or B. These probes can be antibodies or other high-affinity binders. In one embodiment, the probe collection includes at least 1, 2, 5, 10, 50, 100, 500, 1,000, or more antibodies, each of which is capable of binding to the protein product of a different parent sequence selected from Attachments A or B.

It should be understood that the above-described embodiments and the following examples are given by way of illustration, not limitation. Various changes and modifications within the scope of the present invention will become apparent to those skilled in the art from the present description.

### E. EXAMPLES

### Example 1

### Nucleic Acid Array

The tiling sequences depicted in Attachment C were submitted to Affymetrix for custom array design. Affymetrix selected probes for each tiling sequence using its probe-picking algorithm. Non-ambiguous probes with 25 bases in length were selected. Sixty-eight probe-pairs were requested for each tiling sequence with a minimum number of acceptable probe-pairs set to thirty-five. The final array was directed to 4,180 human transcripts and 81 endogenous and exogenous control probes sets. The perfect match probes on the final array are shown in Attachment G and depicted in SEQ ID NOs: 116,338-303,284. The qualifier of each probe, which indicates the corresponding tiling sequence from which the probe was derived, is also provided in Attachment G.

### Example 2

### Nucleic Acid Array Hybridization

10 μg of biotin-labeled sample DNA/RNA is diluted in 1×MES buffer with 100 μg/ml herring sperm DNA and 50 μg/ml acetylated BSA. To normalize arrays to each other and to estimate the sensitivity of the nucleic acid arrays, in vitro synthesized transcripts of control genes are included in each hybridization reaction. The abundance of these transcripts can range from 1:300,000 (3 ppm) to 1:1000 (1000 ppm) stated in terms of the number of control transcripts per total transcripts. As determined by the signal response from these control transcripts, the sensitivity of detection of the arrays can range, for example, between about 1:300,000 and 1:100,000 copies/million. Labeled DNA/RNA are denatured at 99° C. for 5 minutes and then 45° C. for 5 minutes and hybridized to the nucleic array of Example 1. The array is hybridized for 16 hours at 45° C. The hybridization buffer includes 100 mM MES, 1 M [Na⁺], 20 mM EDTA, and 0.01% Tween 20. After hybridization, the cartridge(s) is washed extensively with wash buffer (6×SSPET), for instance, three 10-minute washes at room temperature. The washed cartridge(s) is then stained with phycoerythrin coupled to streptavidin.

12×MES stock contains 1.22 M MES and 0.89 M [Na⁺]. For 1000 ml, the stock can be prepared by mixing 70.4 g MES free acid monohydrate, 193.3 g MES sodium salt and 800 ml of molecular biology grade water, and adjusting volume to 1000 ml. The pH is between about 6.5 and about 6.7. 2× hybridization buffer can be prepared by mixing 8.3 ml of 12×MES stock, 17.7 ml of 5 M NaCl, 4.0 ml of 0.5 M EDTA, 0.1 ml of 10% Tween 20 and 19.9 ml of water. 6×SSPET contains 0.9 M NaCl, 60 mM NaH₂PO₄, 6 mM EDTA, pH 7.4, and 0.005% Triton X-100. In some cases, the wash buffer can be replaced with a more stringent wash buffer, which can be prepared by mixing 83.3 ml of 12×MES stock, 5.2 ml of 5 M NaCl, 1.0 ml of 10% Tween 20 and 910.5 ml of water.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

1. A nucleic acid array comprising polynucleotide probes stably attached to one or more substrate supports, wherein a substantial portion of all polynucleotide probes that are stably attached to the nucleic acid array consists of probes for drug target genes.

2. The nucleic acid array according to claim 1, wherein each said drug target gene is selected from the group consisting of kinase genes, phosphatase genes, protease genes, G-protein coupled receptor genes, nuclear hormone receptor genes, and ion channel genes.

3. The nucleic acid array according to claim 2, wherein said substantial portion of all polynucleotide probes includes at least 25% of all polynucleotide probes that are stably attached to the nucleic acid array.

4. The nucleic acid array according to claim 2, wherein said substantial portion of all polynucleotide probes includes at least 45% of all polynucleotide probes that are stably attached to the nucleic acid array.

5. The nucleic acid array according to claim 1, wherein said substantial portion of all polynucleotide probes includes probes for at least ten kinase genes, probes for at least ten phosphatase genes, probes for at least ten protease genes, probes for at least ten G-protein coupled receptor genes, probes for at least ten nuclear hormone receptor genes, and probes for at least ten ion channel genes.

6. The nucleic acid array according to claim 1, wherein said substantial portion of all polynucleotide probes includes probes for at least fifty kinase genes, probes for at least fifty phosphatase genes, probes for at least fifty protease genes, probes for at least fifty G-protein coupled receptor genes, probes for at least fifty nuclear hormone receptor genes, and probes for at least fifty ion channel genes.

7. The nucleic acid array according to claim 6, wherein said substantial portion of all polynucleotide probes is stably attached to one substrate support.

8. The nucleic acid array according to claim 7, wherein said substantial portion of all polynucleotide probes includes one or more probes for an ADAMTS4 gene.

9. The nucleic acid array according to claim 1, wherein said substantial portion of all polynucleotide probes comprises at least 500 probes, each of which is capable of hybridizing under stringent or nucleic acid array hybridization conditions to a different respective tiling sequence selected from Attachment C, or the complement thereof.

10. The nucleic acid array according to claim 9, wherein said substantial portion of all polynucleotide probes comprises at least 35 probes for each said different respective tiling sequence, or the complement thereof.

11. The nucleic acid array according to claim 10, comprising a mismatch probe for each probe selected from said substantial portion of all polynucleotide probes.

12. The nucleic acid array according to claim 1, wherein said substantial portion of all polynucleotide probes comprises each probe selected from Attachment F, or the complement thereof.

13. A method for identifying or evaluating agents capable of modulating expression profiles of drug target genes, comprising the steps of:
contacting one or more cells with a candidate molecule;
preparing a nucleic acid sample from said one or more cells; and
hybridizing the nucleic acid sample to the nucleic acid array of claim 2 to detect any change in hybridization signals before and after said contacting,
wherein a change in the hybridization signals of a drug target gene is indicative that the candidate molecule is capable of modulating the expression profile of said drug target gene.

14. The method according to claim 13, further comprises the step of administering an effective amount of the candidate molecule to a mammal in need thereof, wherein the candidate molecule is capable of modulating the expression profile of said drug target gene which is selected from the group consisting of a kinase gene, a phosphatase gene, a protease gene, a G-protein coupled receptor gene, a nuclear hormone receptor gene, and an ion channel gene.

15. A method for identifying or evaluating agents capable of modulating expression profiles of drug target genes, comprising the steps of:
administering an agent to a human or animal;
preparing a nucleic acid sample from said human or animal; and
hybridizing the nucleic acid sample to the nucleic acid array of claim 2 to detect any change in hybridization signals before and after said administering,
wherein a change in the hybridization signals of a drug target gene is indicative that the candidate molecule is capable of modulating the expression profile of said drug target gene in said human or animal.

16. A nucleic acid array comprising polynucleotide probes stably attached to one or more substrate supports, wherein a substantial portion of all polynucleotide probes that are stably attached to the nucleic acid array is capable of hybridizing under stringent or nucleic acid array hybridization conditions to corresponding tiling sequences selected from Attachment C, or the complements thereof.

17. The nucleic acid array according to claim 16, wherein said substantial portion of all polynucleotide probes includes at least 100 probes, each of which is capable of hybridizing under stringent or nucleic acid array hybridization conditions to a different corresponding tiling sequence selected from Attachment C, or the complement thereof.

18. The nucleic acid array according to claim 17, wherein said substantial portion of all polynucleotide probes includes at least 45% of all polynucleotide probes that are stably attached to the nucleic acid array.

19. A method, comprising the steps of:
selecting a plurality of polynucleotides, each said polynucleotide capable of hybridizing under stringent or nucleic acid array hybridization conditions to a different respective drug target gene; and
stably attaching said plurality of polynucleotides to one or more substrate supports, wherein a substantial portion of all polynucleotide probes that are stably attached to the nucleic acid array consists of said plurality of polynucleotides.

20. A probe collection comprising:
at least one polynucleotide capable of hybridizing under stringent or nucleic acid array hybridization conditions to a tiling sequence selected from Attachment C, or the complement thereof; or
at least one probe capable of binding to a protein product encoded by a parent sequence selected from Attachments A or B.
